SEQUENCE LISTING (5 sequences)

SEQ ID NO: 1 - protein, 108 aa, Homo sapiens
MKALCLLLLPVLGLLVSSKTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 2 - protein, 23 aa, Homo sapiens
MEEAINERIQEVAGSLIFRAISS

SEQ ID NO: 3 - protein, 27 aa, Homo sapiens
MEEAINERIQEVAGSLIFRAISSIGLE

SEQ ID NO: 4 - protein, 58 aa, Homo sapiens
CQSVTSRGDLATCPRGFAVTGCTCGSACGSWDVRAETTCHCQCAGMDWTGARCCRVQP

SEQ ID NO: 5 - protein, 247 aa, Homo sapiens
PWPGVPGGRSKTSGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0009987 A9
(43) Pub. Date: Jan. 13, 2022

(54) HUMAN RESISTIN COMPOSITIONS AND METHODS OF TREATING TLR4-MEDIATED DISEASE AND INFECTION

(71) Applicant: THE REGENTS OF THE UNIVERSITY OF CALIFORNIA, Oakland, CA (US)

(72) Inventors: Meera G. NAIR, Riverside, CA (US); Jessica JANG, Riverside, CA (US); Gang CHEN, Riverside, CA (US)

(21) Appl. No.: 16/498,365

(22) PCT Filed: Apr. 12, 2018
(86) PCT No.: PCT/US18/27387

§ 371 Date: Sep. 26, 2019

Related U.S. Application Data
(60) Provisional application No. 62/485,881, filed on Apr. 14, 2017.

(51) Int. Cl. C07K 14/575; A61P 31/00

(52) U.S. Cl. CPC C07K 14/575; A61K 38/00; C07K 2319/30; A61P 31/00

(57) ABSTRACT

Compositions and methods for treating, mitigating, or preventing a Toll-like receptor (4) (TLR4)-mediated infection or disease in a host cell or a subject include administration of a resistin protein composition selected from full length resistin protein, a resistin protein fragment, or a fusion protein of resistin and an immunoglobulin G protein to the host cell or the subject having or likely to be exposed to a TLR4-mediated infection or disease.

### CROSS-REFERENCE TO RELATED APPLICATION(S)

The present application claims priority to and the benefit of U.S. Provisional Application Ser. No. 62/485,881 filed on Apr. 14, 2017, entitled “Human Resistin Compositions and Methods of Treating TLR4-Mediated Disease and Infection,” the entire content of which is incorporated herein by reference.

### INCORPORATION BY REFERENCE

The instant application contains a Sequence Listing which has been submitted electronically in ASCII format and is hereby incorporated by reference in its entirety. Said ASCII copy, created on Apr. 10, 2018, is named 134094SEQLISTING.txt and is 4,623 bytes in size.

### BACKGROUND

The Toll-like receptor 4 (TLR4) is activated in many pathogenic infections as well as cellular stress caused by physical trauma. Examples of TLR4-mediated infections or diseases include sepsis, necrotizing enterocolitis (NEC), acute respiratory distress syndrome (ARDS), rheumatoid arthritis, influenza, and traumatic injury. The pathogen-associated lipopolysaccharide (LPS), also known as endotoxin, is a main component of gram negative bacterial cell walls and activates by binding TLR4. LPS binding to TLR4 induces an NF-κB-dependent inflammatory cascade resulting in excessive production of TNFα and IL-6. These pro-inflammatory cytokines are initially beneficial in bacterial killing, but they eventually damage the host's cells and tissues. For example, excessive production of TNFα causes endothelial cell injury, which may eventually to vascular permeability, low blood pressure, and organ failure.

Recent studies have targeted the TLR4 inflammatory pathway as a potential treatment of sepsis and necrotizing enterocolitis (NEC). However, clinical trials with anti-TLR4 antibodies or TLR4 antagonists have not been successful and sepsis is currently treated with antibiotics and supportive care.

### SUMMARY

Aspects of embodiments of the present disclosure relate to the use of a recombinant human resistin protein (hRetn) for treating, mitigating, or preventing TLR4-mediated infections and diseases. Examples of TLR4-mediated infections or diseases include inflammatory disorders, as well as, for example, sepsis, acute respiratory distress syndrome (ARDS), necrotizing enterocolitis, autoimmune diseases, Crohn's disease, celiac disease, ulcerative colitis, rheumatoid arthritis, cardiovascular disease including myocardial infarction, epilepsy, gram negative bacterial infections, aspergillosis, periodontal disease, Alzheimer's disease, cigarette smoke mediated lung inflammation, viral hepatitis (including hepatitis C virus hepatitis), alcoholic hepatitis, and insulin resistance in adipocytes. TLR4-mediated disorders also include ischemic injury and traumatic injury to the heart, liver, lung, kidney, intestine, brain, eye and pancreas.

In addition to the full length hRetn protein (SEQ ID NO: 1), compositions of a recombinant hRetn also include a fragment of the full-length resistin protein that is capable of binding TLR4 or decreasing a TLR4-mediated inflammatory response.

In some embodiments, a fragment of the full-length resistin includes an N-terminal fragment or a C-terminal fragment of the human resistin protein of SEQ ID NO: 1. The N-terminal fragment may include at least 84 consecutive amino acids of residues 24 to 108 of the human resistin protein of SEQ ID NO: 1 or homologs thereof. In some embodiments, the N-terminal fragment of includes at least residues 24 to 46 (SEQ ID NO: 2) or at least residues 24 to 50 (SEQ ID NO: 3) or homologs thereof. The C-terminal fragment may include residues 51 to 108 (SEQ ID NO: 3).

Compositions of a recombinant hRetn include a fusion protein of a full-length human resistin protein or a fragment of the human resistin protein fused with an immunoglobulin G (IgG) protein. Examples of recombinant hRetn fusion protein include hRetn (SEQ ID NO:1), an N-terminal fragment of hRetn, or a C-terminal fragment of hRetn each fused with a fragment crystallizable (Fc) region of the IgG (Fc IgG) protein having an amino acid sequence of SEQ ID NO: 5.

According to some embodiments of the present disclosure, a method of treating a Toll-like receptor 4 (TLR4)-mediated infection or disease in a host cell or in a subject having the TLR4-mediated infection or disease includes administering to the host cell or the subject an effective amount of a composition including a full length resistin protein, a resistin protein fragment, a fusion protein of a resistin protein or a fragment thereof and an immunoglobulin G protein, a fusion protein of an N-terminal or C-terminal resistin protein fragment and an immunoglobulin G (IgG) protein, a homolog thereof, a derivative thereof, or a salt thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The accompanying drawings, together with the specification, illustrate example embodiments of the present disclosure, and, together with the description, serve to explain the principles of the present disclosure.

FIG. 1A outlines the experimental design used for an LPS-induced sepsis model in transgenic mice that express human resistin protein (hRETNTg⁺) and control mice (hRETNTg−), according to embodiments of the present disclosure.

FIG. 1B is a graph showing the amount of hRetn serum levels in hRETNTg+(Tg+) mice and a second transgenic mouse model expressing human resistin protein (hResistin) (Tg2+) mice as measured by ELISA, according to embodiments of the present disclosure.

FIG. 1C is a graph plotting the survival rate of C57BL/6 mice (red dot and corresponding red line), control hRETNTg− mice (Tg−)(black square and corresponding black line), Tg+ mice (blue triangle and corresponding blue line), and Tg2+ mice (green diamond and corresponding green line) evaluated following high dose LPS (12 mg/kg) injection, according to embodiments of the present disclosure.

FIG. 1D is a graph showing the rectal body temperature (° F.) measured at 0 and 6 hours post high dose LPS injection in hRETNTg− (Tg−) mice and hRETNTg⁺ (Tg+) mice at 0 hour and 18 hour post LPS injection, according to embodiments of the present disclosure.

FIG. 1E shows a series of graphs showing the percentage of live (% live) peritoneal exudate cells (PEC) including macrophages, neutrophils, eosinophils, and monocytes, as indicated, that were recovered and analyzed by flow cytometry from hRETNTg− (Tg−) mice and hRETNTg⁺ (Tg+) mice without challenge of LPS (˜12 hr) and with high dose LPS challenge, according to embodiments of the present disclosure.

FIG. 1F is table showing the raw data of 33 cytokines as indicated from unchallenged (Naïve) hRETNTg− (Tg−) mice and hRETNTg⁺ (Tg+) mice and LPS-challenged C57BL/6, Tg−, and Tg+ mice, according to embodiments of the present disclosure.

FIG. 1G is a heatmap with color key using data from FIG. 1F for the indicated serum cytokines that were induced 6 hours following LPS treatment in hRETNTg− (Tg−) mice and hRETNTg⁺ (Tg+) mice, according to embodiments of the present disclosure.

FIG. 1H is a series of bar graphs showing levels (in pg/mL) of the indicated pro-inflammatory and anti-inflammatory cytokines in C57BL/6 (B6), hRETNTg− (Tg−) and hRETNTg⁺ (Tg+) mice without challenge of LPS (˜12 hr) and with high dose LPS challenge (6 hours), with the data presented as mean±SEM where n is 7 to 12 for survival, n is 3 to 5 for other parameters, and the data is representative of 3 separate experiments, according to embodiments of the present disclosure.

FIG. 11 is a table listing the chromosomal insertion sites for hRETN in the hRETNTg+ (Tg+) mice and the second transgenic hRETNTg2+ (Tg2+) mice, according to embodiments of the present disclosure.

FIG. 2A outlines the experimental design used to test the role of hRetn protein in LPS-induced sepsis, according to embodiments of the present disclosure.

FIG. 2B is a graph showing the survival rate (% Survival) over time (hours) in C57BL/6 mice treated with PBS (blue line) or with 500 ng/mouse hRetn protein (red line) prior to LPS injection, according to embodiments of the present disclosure.

FIG. 2C is a graph showing the rectal body temperature (° F.) in C57BL/6 mice unchallenged (naïve) C57BL/6 mice or C57BL/6 mice 6 hours after LPS or hRetn protein and LPS, according to embodiments of the present disclosure.

FIG. 2D is a graph showing the amount of Evan's blue dye (ng/mg tissue) representing lung vascular permeability in unchallenged (naïve) C57BL/6 mice or C57BL/6 mice 6 hours after LPS or hRetn protein and LPS, according to embodiments of the present disclosure.

FIG. 2E is a series of bar graphs showing the amount of the indicated cytokine (pg/mL of IL-6, TNFα, IFNγ, and IL-10) in C57BL/6 mice 6 hours after LPS or hRetn protein and LPS, according to embodiments of the present disclosure.

FIG. 2F is a series of bar graphs showing the percentage of live (% live) peritoneal exudate cells (PEC) including macrophages, neutrophils, eosinophils, and monocytes, as indicated, that were recovered and analyzed by flow cytometry from C57BL/6 mice unchallenged (naïve) C57BL/6 mice or C57BL/6 mice 6 hours after LPS or hRetn protein and LPS, according to embodiments of the present disclosure.

FIG. 3A outlines the experimental design in hRETNTg⁺ mice infected with Nippostrongylus brasiliensis (Nb) for 14 days followed by LPS challenge together with a bar graph showing the amount of hRetn protein measured in the serum of naïve (n) uninfected hRETNTg⁺ mice or Nb-infected (INF) mice at −48 hours (left) or 6 hours (right) post LPS (+LPS) injection, from two separate experiments each with a sample size (n) of 3 to 4, according to embodiments of the present disclosure.

FIG. 3B is a graph showing the survival rate (% Survival) over time (hours) after LPS injection in hRETNTg⁻ (Tg−) mice that received injection of LPS (blue line) or infection with Nb followed by LPS injection (green line) or in hRETNTg⁺ (Tg+) mice that received injection with LPS (red line) or infection with Nb followed by LPS injection (black triangle and corresponding black line), from two separate experiments each with a sample size (n) of 6 to 10, according to embodiments of the present disclosure.

FIG. 3C is a series bar graphs showing the amount of the indicated cytokine (pg/mL of IL-6, IL-10, MCP1, IFNγ, and TNFα) from Nb-infected and LPS-challenged hRETNTg⁻ (Tg−) and hRETNTg⁺ (Tg+) mice, from two separate experiments each with a sample size (n) of 3 to 4, according to embodiments of the present disclosure.

FIG. 3D is a bar graph showing the amount of total cells measured by flow cytometry for each indicated peritoneal cell type (macrophages (Macs), monocytes (Mono), neutrophils (Neut), and eosinophils (Eos)) in hRETNTg⁻ (Tg−) and hRETNTg⁺ (Tg+) mice recovered at 48 hours post-LPS challenge and assayed directly ex vivo, from two separate experiments each with a sample size (n) of 3 to 4, according to embodiments of the present disclosure.

FIG. 3E is an image of a representative Western blot (left panel) of phosphorylated STAT3 (pSTAT3), phosphorylated TBK-1 (pTBK-1), and IκBα with endogenous β-actin as a control in the peritoneal cells from hRETNTg⁻ (Tg−) and hRETNTg⁺ (Tg+) mice recovered at 48 hours post-LPS challenge together with bar graphs (right panel) showing the quantified band density (Relative Expression) for each of pSTAT3, TBK-1, and IκBα for 3 mice per group, according to embodiments of the present disclosure.

FIG. 4A is a series of graphs measuring the amount of mRNA expression (RPKM) for cyclic adenylate associated protein 1 (Cap1) and TLR4 in naïve or day 7 Nb-infected lungs from hRETNTg⁻ (Tg−) and hRETNTg⁺ (Tg+) mice as measured by RNA sequencing, according to embodiments of the present disclosure.

FIG. 4B is a series of graphs of flow cytometric analysis of day 7 Nb-infected Tlr4+/+ and Tlr4−/− lung cells after incubation with or without hRetn showing hRetn binding to monocytes (Ly6C+ cells) is TLR4-dependent, according embodiments of the present disclosure.

FIG. 4C is a bar graph showing the amount (%) of hRetn protein bound to monocytes, alveolar macrophages (Alv. Macs), and neutrophils in day 7 Nb-infected Tlr4+/+(black bars) and Tlr4−/− (blue bars) lung cells from the hRETNTg⁻ (Tg−) and hRETNTg⁺ (Tg+) mice was measured as frequency of hRetn-bound cells, according embodiments of the present disclosure.

FIG. 4D is a graph showing the change in (A) Mean Fluorescence Intensity (ΔMFI) measured in monocytes, alveolar macrophages, and neutrophils in day 7 Nb-infected Tlr4+/+(black bars) and Tlr4−/− (blue bars) lung cells from the hRETNTg⁻ (Tg−) and hRETNTg⁺ (Tg+) mice, according embodiments of the present disclosure.

FIG. 4E is a bar graph showing the percentage of live (% live) monocytes, alveolar macrophages (Alv. Macs), and neutrophils, as indicated, found in lung cells from the hRETNTg⁻ (Tg−) and hRETNTg⁺ (Tg+) mice, according to embodiments of the present disclosure.

FIG. 4F is a pie chart of the proportion cell types bound by hRetn, according to embodiments of the present disclosure.

FIG. 4G is an image of anti-His and anti-hRetn Western blots from a pull-down assay with His-tagged TLR4, His-CAP1 or His-MBP as indicated, in both E. coli and 293T derived hRetn, according to embodiments of the present disclosure.

FIG. 5A is a schematic depicting the calculated structure of the human resistin protein (hRetn) shown in green based on the structure of mouse resistin protein (mRetn) shown in cyan, according to embodiments of the present disclosure.

FIG. 5B is an image of a structural model (far left upper and lower panels) of hRetn with the N-terminus in blue (N-Retn) and the C-terminus in green (C-Retn) and TLR4 in red showing that hRetn binds in the same binding pocket of MD2, the adaptor protein for LPS (shown in white), according to embodiments of the present disclosure.

FIG. 5C is a schematic depicting the calculated molecular interactions between the N-terminal helical trimer of hRetn (shown in cyan, red, yellow) and the TLR4 monomer (shown in blue), according to embodiments of the present disclosure.

FIG. 5D is a pie chart showing the proportion of hRetn-bound cells in human peripheral blood mononuclear cells (PBMCs), according to embodiments of the present disclosure.

FIG. 5E is a graph of a side scatter (SSC) flow cytometric analysis of LPS-bound CD14⁺ CD11 b⁺ monocytes in PBS alone (without LPS), LPS alone, or prior incubation with hRetn followed by LPS, according to embodiments of the present disclosure.

FIG. 5F is a graph showing the statistical analysis of the data in FIG. 5E for amount (%) of LPS-bound to the CD14⁺ CD11b⁺ monocytes, according to embodiments of the present disclosure.

FIG. 5G is a graph showing the amount (pg/mL) of secreted TNFα measured in PBMCs treated with PBS (without LPS), LPS, or hRetn followed by LPS, according to embodiments of the present disclosure.

FIG. 5H is a schematic depicting the primary sequence of the synthesized hRetn N-terminal peptide and a circular dichroism (CD) spectrum of the hREtn N-terminal peptide measured at 100 μM, according to embodiments of the present disclosure.

FIG. 5I is a series of images of anti-His and anti-Flag Western blots from a pull-down assay with His-tagged TLR4 incubated with control buffer or hRetn N-terminal peptide (N-pep), followed by incubation with 293T cell-derived full length Flag-tagged hRetn, according to embodiments of the present disclosure.

FIG. 5J is a graph showing the amount (%) of LPS bound in human PBMCs incubated in PBS alone, with LPS, with hRetn protein prior to LPS, or with N-pep prior to LPS, according to embodiments of the present disclosure.

FIG. 5K is a graph showing the level of mean fluorescence intensity (MFI) correlating to the cell surface expression of MD2 (LPS adaptor protein) and TLR4 in human PBMCs incubated in PBS alone, with LPS, with hRetn protein prior to LPS, or with N-pep prior to LPS, according to embodiments of the present disclosure.

FIG. 5L is a graph showing the amount (pg/mL) of secreted TNFα measured in PBMC treated with PBS alone, with LPS, with hRetn protein prior to LPS, or with N-pep prior to LPS, according to embodiments of the present disclosure.

FIG. 6A is an image of a representative Western blot (left panel) and a series of band density graphs (right panels) showing the relative expression levels of phosphorylated STAT3 (pSTAT3), phosphorylated TBK-1, and IκBα, relative to β-actin in peritoneal exudate cells (PECs) collected from 6 to 8 week old naïve hRETNTg⁺ or hRETNTg⁻ mice having a Tlr4^{+/+} or Tlr4^{-/-} background and analyzed directly ex vivo, according to embodiments of the present disclosure.

FIG. 6B is a series of bar graphs showing the percentage of live (% live) peritoneal exudate cells (PEC) including macrophages, neutrophils, eosinophils, and monocytes, as indicated, that were recovered and analyzed by flow cytometry from 6 to 8 week old naïve hRETNTg⁺ or hRETNTg⁻ mice having a Tlr4^{+/+} or Tlr4^{-/-} background and analyzed directly ex vivo, according to embodiments of the present disclosure.

FIG. 6C is a series of graphs measuring the amount (pg/ML) of human TNF (far left graph), the amount (%) of TNF inhibition (middle left graph), the amount (pg/ML) of human IL-10 (middle right graph) or the amount (%) of IL-10 inhibition (far right graph) in human PBMCs pre-treated with a DMSO control, TBK1 inhibitor or a STAT3 inhibitor, followed by addition of PBS (black bars) or hRetn (blue bars) prior to LPS treatment, according to embodiments of the present disclosure.

FIG. 7A depicts an expression vector pMT/BiP for expression of human resistin protein and human resistin fragments fused with human IgG1 Fc fragment, for example, the full-length human resistin protein (hRetn) (with an N-terminal fragment in blue and a C-terminal fragment in green) is fused with human IgG1 Fc (SEQ ID NO: 5) (shown in magenta), a C-terminal fragment of hRetn protein (SEQ ID NO: 4) (shown in green) is fused with human IgG1 Fc (SEQ ID NO: 5) (shown in magenta), and the N-terminal fragment of hRetn protein (SEQ ID NO: 3) (shown in blue) is fused with human IgG1 Fc (SEQ ID NO: 5) (shown in magenta), according to embodiments of the present disclosure.

FIG. 7B is a Western blot showing stable transfection of hIgG (human IgG) and hRetn-hIgG (full-length human resistin protein fused with human IgG), according to embodiments of the present disclosure.

FIG. 7C shows graphs of inhibition of LPS-induced TNFα, IL-6, or promotion of IL-10 expression, as indicated, in primary macrophage culture in the presence of supernatant from hRetn-hIgG transfected cells compared to supernatant from hIgG transfected cells, according to embodiments of the present disclosure.

### DETAILED DESCRIPTION

The innate immune receptor Toll-like receptor 4 (“TLR4”) has been recognized to be the receptor on hematopoietic and non-hematopoietic cells for lipopolysaccharide, “LPS” as well as a variety of endogenous molecules that are released within the body during inflammatory diseases or infections.

As TLR4 is implicated in many pathogenic and inflammatory disorders, regulators are needed that treat, mitigate, and/or prevent the damaging and sometimes fatal effects of TLR4-mediated infections or diseases.

The human resistin protein (hRetn) is an immune cell-derived protein that has been shown to be highly elevated in TLR4-mediated infections including sepsis and helminth (e.g., hookworm) infections, as well as acute respiratory distress syndrome (ARDS), rheumatoid arthritis, influenza, and traumatic injury.

Aspects of embodiments of the present disclosure are directed to methods of treating TLR4-mediated infections and diseases using recombinant hRetn proteins and derivatives. As disclosed herein, both recombinant hRetn proteins, hRetn protein fragments, and hRetn fusion immunoglobulin G (IgG) proteins mitigate lipopolysaccharide (LPS)-induced septic shock.

Accordingly, embodiments of the present disclosure include a method of treating a TLR4-mediated infection or disease in a host cell or in a subject by administering one of the recombinant hRetn proteins, hRetn protein fragments, or hRetn fusion IgG proteins of the present disclosure to the host cell or the subject having a TLR4-mediated infection or disease. Alternatively or additionally, in other embodiments of the present invention, a method of mitigating an expected exposure or incidence of a TLR4-mediated infection or disease includes administering one of the recombinant hRetn proteins, hRetn protein fragments, or hRetn fusion IgG proteins of the present disclosure to the host cell or the subject prior to the expected exposure or incidence.

Compositions according to embodiments of the present disclosure include a resistin protein, a resistin protein fragment, a fusion protein made of a resistin protein, an N-terminal resistin protein fragment, or a C terminal resistin protein fragment fused with immunoglobulin G (IgG) protein, homologs thereof, a derivative thereof, or a salt thereof. A composition according to embodiments of the present disclosure includes the active domain of resistin (e.g., SEQ ID NO: 2), homologs thereof, and derivatives thereof. As disclosed herein, the active domain of resistin block TLR4-LPS interaction.

As used herein, “treating,” “treat,” “treatment,” and like terms refer to a change in a TLR4-mediated inflammatory response including a decrease in a pro-inflammatory cytokine (e.g., TNFα or IL-6) as a result of administration of a recombinant hRetn composition according to the present disclosure.

As used herein, “mitigate,” “mitigating,” and like terms refer to a decrease in or an absence of an expected or possible TLR4-mediated inflammatory response in a host cell or subject based upon exposure to an agent known to induce a TLR4-mediated infection or disease. An absence of an unexpected or possible TLR4-mediated inflammatory response in a host cell or subject based upon exposure to an agent known to induce a TLR4-mediated infection or disease may also be referred to as “preventing.”

As used herein, an “effective amount” refers to an effective concentration for a host cell or subject of a recombinant hRetn composition for inducing treatment of the host cell or subject or mitigating or preventing the incidence of a TLR4-mediated infection or disease.

As used herein, TNFα refers to the tumor necrosis factor protein.

As used herein, NFκB refers to nuclear factor kappa-light chain enhancer of activated B cells protein.

As used herein, IκB refers to inhibitor of kappa B protein.

As used herein, IFNγ refers to type II interferon protein.

As used herein, IL-6, IL-10, IL-1a refer to interleukin 6, interleukin 10, and interleukin 1 alpha proteins, respectively.

As used herein, MCP1 refers to monocyte chemoattractant protein-1.

Abbreviations for amino acids are used throughout this disclosure and follow the standard nomenclature known in the art. For example, as would be understood by those or ordinary skill in the art, Alanine is Ala or A; Arginine is Arg or R; Asparagine is Asn or N; Aspartic Acid is Asp or D; Cysteine is Cys or C; Glutamic acid is Glu or E; Glutamine is Gln or Q; Glycine is Gly or G; Histidine is His or H; Isoleucine is Ile or I; Leucine is Leu or L; Lysine is Lys or K; Methionine is Met or M; Phenylalanine is Phe or F; Proline is Pro or P; Serine is Ser or S; Theonine is Thr or T; Tryptophan is Trp or W; Tyrosine is Tyr or Y; and Valine is Val or V.

As used herein “homologs” and “homology” in the context of a referenced amino acid sequence include proteins homologs sharing 80%, 85%, 90%, 95%, 96%, 97%, 98%, or 99% amino acid sequence identity to the referenced amino acid sequence or the protein homolog shares 100%, 99%, 98%, 97%, 96%, or 95% amino acid similarity to the referenced amino acid sequence. A homolog having 100% amino acid similarity is also referred to as an analog or derivative as disclosed herein.

In some embodiments of the present disclosure, the resistin protein fragment for treating a TLR4-mediated infection or disease is made of at least one of Chain M, Chain H, or Chain C of the human resistin protein (hRetn) having an amino acid sequence of SEQ ID NO: 1 (MKALCLLLLPVLGLLVSSKTLCSMEEAINERIQEVAGSLIFRAISSIGLECQSVTSRGDL ATCPRGFAVTGCTCGSACGSWDVRAETTCHCQCAGMDWTGARCCRVQP). In some embodiments, the Chain M, Chain H, or Chain C is in monomeric or trimeric form.

A composition of an N-terminal resistin fragment includes any recombinant fragment of the full length human resistin protein (hRetn) (SEQ ID NO: 1) having at least residues 24 to 46 (SEQ ID NO: 2) (MEEAINERIQEVAGSLIFRAISS), or homologs thereof having homology to SEQ ID NO: 2. In some embodiments, the N-terminal resistin fragment is made of at least residues 24 to 50 (SEQ ID NO:3) (MEEAINERIQEVAGSLIFRAISSIGLE), or homologs thereof having homology to SEQ ID NO: 3.

A composition of a C-terminal resistin fragment includes any recombinant fragment of the full length human resistin protein (hRetn) (SEQ ID NO: 1) having at least residues 51 to 108. (SEQ ID NO: 4) (CQSVTSRGDLATCPRGFAVTGCTCGSACGSWDVRAETTCHCQCAGMDWTGARCC RVQP), or homologs thereof having homology to SEQ ID NO: 4.

A composition of a hRetn protein fused with an IgG protein (“hRetn-IgG”) includes any recombinant hRetn protein or hRetn protein fragment fused to an immunoglobulin G (IgG) protein. For example the IgG protein may include only the Fc (crystallizable fragment) of the IgG protein. For example, a hRetn-IgG composition may include each of the hRetn of SEQ ID NO: 1, the N-terminal hRetn of SEQ ID NO: 2, the N-terminal hRetn of SEQ ID NO: 3, or the hRetn of SEQ ID NO: 4 fused with Fc IgG of SEQ ID NO: 5

(PWPGVPGGRSKTSGSEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK).

According to embodiments of the present disclosure, methods for treating, mitigating, or preventing a Toll-like receptor 4 (TLR4)-mediated infection or disease in a host cell or in a subject having or likely to be exposed to the TLR4-mediated infection or disease, includes administering to the host cell or the subject an effective amount of a composition including any recombinant human resistin protein (hRetn) as disclosed herein. Examples of recombinant hRetn include a full length resistin protein, a resistin protein fragment, a fusion protein comprising a resistin protein or a fragment thereof and an immunoglobulin G protein, a fusion protein of an N-terminal or C-terminal resistin protein fragment and an immunoglobulin G (IgG) protein, homologs thereof, a derivative thereof, or a salt thereof. In some embodiments, the resistin protein fragment is the active domain N-terminal fragment of resistin (SEQ ID NO: 2) that blocks TLR4-LPS interaction.

Methods for treating, mitigating, or preventing a TLR4-mediated infection or disease in a host cell or subject includes administering an effective amount of a recombinant hRetn of the present disclosure including homologs, derivatives, and salts thereof. With reference to FIG. 2B, mice administered recombinant hRetn protein prior to being exposed to LPS were protected against an otherwise lethal dose of LPS.

Recombinant hRetn compositions for treating, mitigating, or preventing a TLR4-mediated infection or disease include a composition of an N-terminal resistin fragment having at least residues 24 to 46 (SEQ ID NO: 2), or homologs thereof having homology to SEQ ID NO: 2. In some embodiments, the N-terminal resistin fragment is made of at least residues 24 to 50 (SEQ ID NO:3) or homologs thereof having homology to SEQ ID NO: 3. With reference to FIGS. 5J-5L, an N-terminal resistin fragment of SEQ ID NO: 2 (“N-pep”) abrogated the binding of LPS in human PMBCs similar to full-length hRetn.

Recombinant hRetn compositions for treating, mitigating, or preventing a TLR4-mediated infection or disease also include a composition of a C-terminal resistin fragment having at least residues 51 to 108. (SEQ ID NO: 4) of hRetn (SEQ ID NO:1) or homologs thereof having homology to SEQ ID NO: 4.

A composition of a hRetn protein fused with an IgG protein (“hRetn-IgG”) includes any recombinant hRetn protein or hRetn protein fragment fused to an immunoglobulin G (IgG) protein. For example the IgG protein may include only the Fc (crystallizable fragment) of the IgG protein. For example, a hRetn-IgG composition may include one of the hRetn full length protein of SEQ ID NO: 1, the N-terminal hRetn protein fragment of SEQ ID NO: 2, the N-terminal hRetn protein fragment of SEQ ID NO: 3, or the C-terminal hRetn protein fragment of SEQ ID NO: 4 fused with Fc IgG of SEQ ID NO: 5.

With reference to FIG. 7A, a vector construct (pMT/BiP) expressing the hRetn fusion IgG proteins is shown depicting the recombinant full length hRetn-IgG Fc fusion, an N-terminal hRetn-IgG Fc fusion, and a C-terminal hRetn-IgG fusion. FIG. 7B shows the full length hRetn-IgG fusion expressed in Drosophila S2 cell lines, and FIG. 7C shows that the full length hRetn-IgG fusion decreases LPS-induced levels of TNFα and IL-6 and promotes IL-10 expression in macrophages.

As used herein, the term “derivative” or “analog” are used interchangeably, and refer to a deletion, addition or substitution of one or more amino acid residues in a resistin protein or resistin protein fragment (e.g., a resistin peptide). When preparing derivatives or analogs obtained by substitution of amino acid residues, it is important that the substitutions be selected from those which cumulatively do not substantially change the volume, hydrophobic-hydrophilic pattern and charge of the corresponding portion of the unsubstituted parent peptide. Thus, a hydrophobic residue may be substituted with a hydrophilic residue, or vice-versa, as long as the total effect does not substantially change the volume, hydrophobic-hydrophilic pattern and charge of the corresponding unsubstituted parent peptide, e.g., as long as the TLR4 binding is maintained.

It should be understood that other modifications of the peptides and analogs thereof are also contemplated by embodiments of the present disclosure. Thus, the resistin protein, resistin protein fragment, peptide or analog of the present disclosure includes a “chemical derivative” thereof which retains at least a portion of the function of the resistin protein which permits its utility in modulating the activity of a TLR4 receptor protein in response to ligand activation.

In some embodiments, the resistin protein, resistin protein fragments, or derivatives thereof are in the form of pharmaceutical salts. As used herein, the term “salts” refers to both salts of carboxyl groups and to acid addition salts of amino groups of the resistin protein, resistin fragment, or resistin peptide molecule. Salts of a carboxyl group may be formed by means known in the art and include inorganic salts, for example, sodium, calcium, ammonium, ferric or zinc salts, and the like, and salts with organic bases such as those formed for example, with amines, such as triethanolamine, arginine, or lysine, piperidine, procaine, and the like. Acid addition salts include, for example, salts with mineral acids such as, for example, hydrochloric acid or sulfuric acid, and salts with organic acids, such as, for example, acetic acid or oxalic acid. Such derivatives and salts are preferably used to modify the pharmaceutical properties of the resistin protein, resistin protein fragment, or resistin peptide for stability and solubility.

In some embodiments of the present disclosure, the composition including the resistin protein, resistin protein fragment, a fusion protein of a resistin protein or resistin protein fragment fused with IgG Fc protein, a homolog thereof, a derivative thereof, or a salt thereof also includes a pharmaceutical carrier.

As used herein, the term “pharmaceutically acceptable carrier” or “pharmaceutical carrier” are used interchangeably to refer to any pharmaceutically acceptable means to mix and/or deliver the resistin composition to a subject. For example, a “pharmaceutically acceptable carrier” refers to a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each pharmaceutical carrier must be “acceptable” in the sense of being compatible with the other ingredients of the formulation and is compatible with administration to a subject, for example a human. For the clinical use of the methods according to some embodiments of the present disclosure, resistin compositions according to some embodiments are formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound according to some embodiments of the disclosure in combination with one or more pharmaceutically acceptable ingredients. The pharmaceutical carrier may be in the form of a solid, semi-solid or liquid diluent, cream or a capsule. These pharmaceutical preparations are a further object according to some embodiments of the disclosure. Usually, the amount of active compounds is between 0.1-95% by weight of the preparation, for example, between 0.2-20% by weight in preparations for parenteral use and for example, between 1 and 50% by weight in preparations for oral administration.

According to embodiments of the present disclosure, a resistin composition as disclosed herein (a resistin protein, a resistin protein fragment/resistin peptide, a fusion protein of resistin protein or a resistin protein fragment fused with immunoglobulin G (IgG), a homolog thereof, a derivative thereof, or a salt thereof may be is used to treat, mitigate, and/or prevent any disease or infection mediated by TLR4 receptor. Non-limiting examples of TLR4-mediated infections or diseases include inflammatory disorders, as well as specifically, sepsis, acute respiratory distress syndrome (ARDS), necrotizing enterocolitis, autoimmune diseases, Crohn's disease, celiac disease, ulcerative colitis, rheumatoid arthritis, cardiovascular disease including myocardial infarction, epilepsy, gram negative bacterial infections, aspergillosis, periodontal disease, Alzheimer's disease, cigarette smoke mediated lung inflammation, viral hepatitis (including hepatitis C virus hepatitis), alcoholic hepatitis, insulin resistance in adipocytes, and others. See, for example, U.S. Patent Application Publication No. US2008-0311112-A1, published Dec. 18, 2008 and J Immunol. 2015 Feb. 1; 194(3): 855-860. doi:10.4049/jimmunol.1402513, the entire contents of both of which are herein incorporated by reference. The present disclosure may also be used, in non-limiting embodiments, to treat post traumatic conditions, including ischemic injury and traumatic injury to the heart, liver, lung, kidney, intestine, brain, eye and pancreas.

In some embodiments of the present disclosure, a resistin composition as disclosed herein (a resistin protein, a resistin protein fragment/resistin peptide, a resistin-IgG fusion protein, a homolog thereof, a derivative thereof, or salt thereof), is used to treat sepsis, rheumatoid arthritis, influenza, traumatic brain injury, or acute respiratory distress syndrome (ARDS). The role of TLR4 in sepsis is disclosed in the Examples herein. The roles of TLR4 in rheumatoid arthritis, influenza, traumatic brain injury, and acute respiratory distress syndrome (ARDS), are disclosed respectively in Kiyeko et al., EJI, 2016, 46:2629-2638, Q. M. Nhu et al., Mucosal Immunology, 2010, 3:29-39 and WO 2013/148072, Zhang et al., Neurochemistry International, 2014, 75: 11-18, and J Immunol. 2015 February 1; 194(3): 855-860. doi:10.4049/jimmunol.1402513, the entire contents of all of which are herein incorporated by reference.

The following Examples are presented for illustrative purposes only, and do not limit the scope or content of the present application.

### EXAMPLES

### Example 1

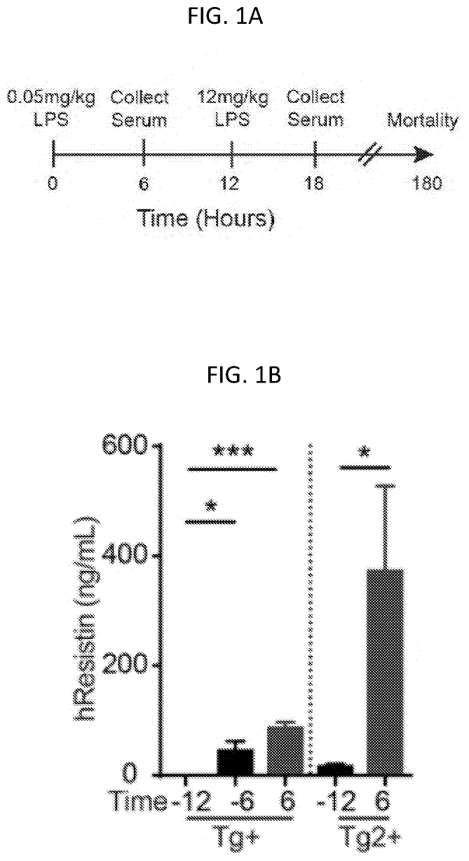

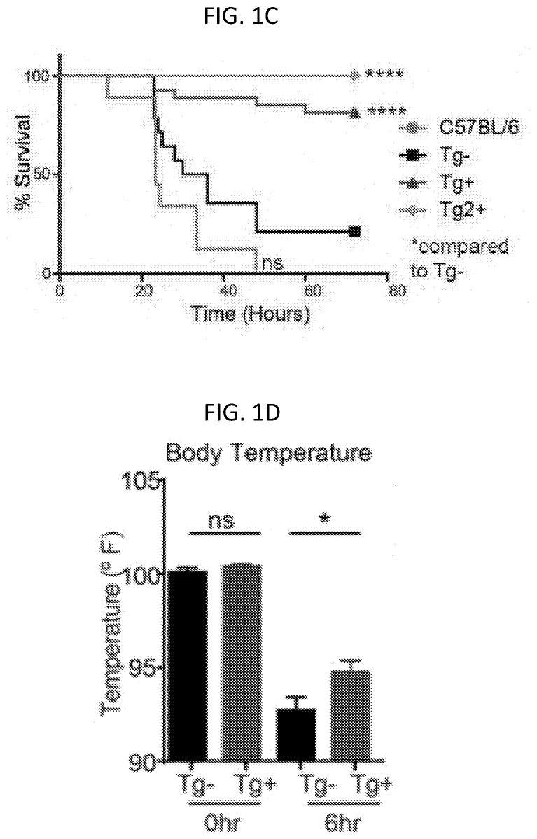

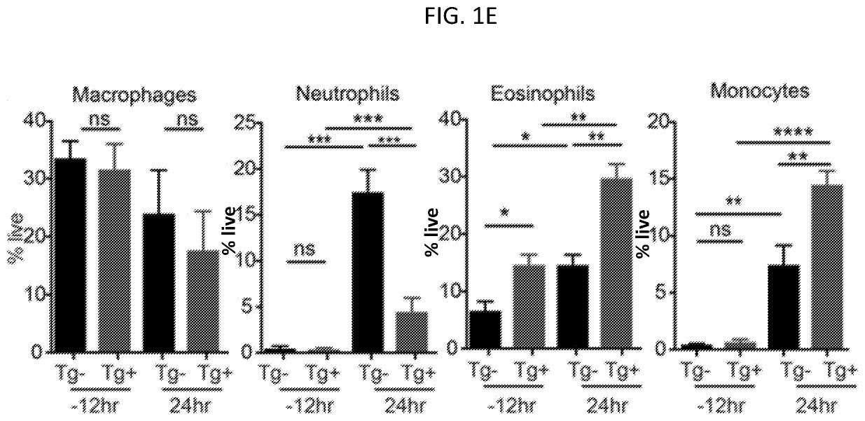

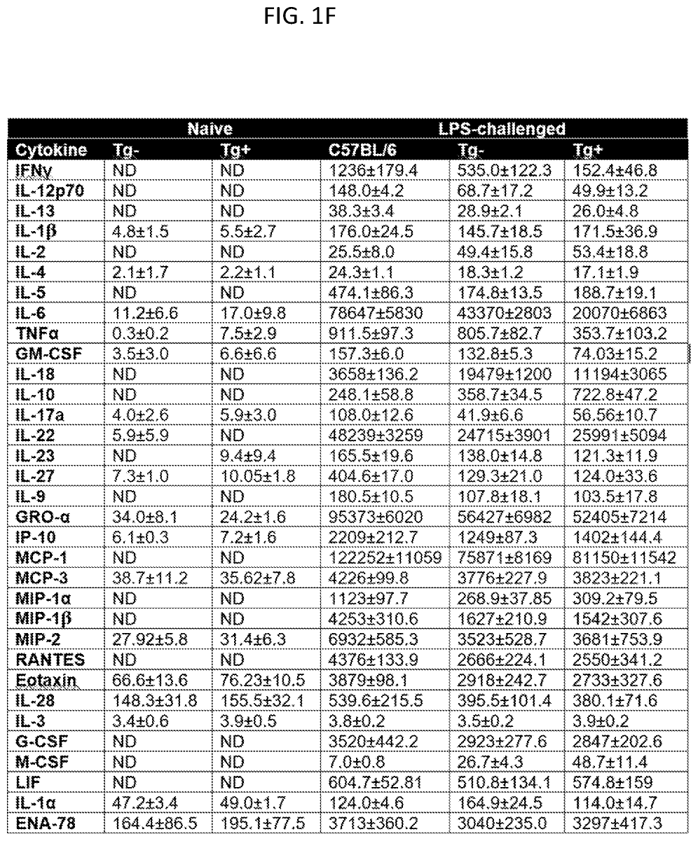

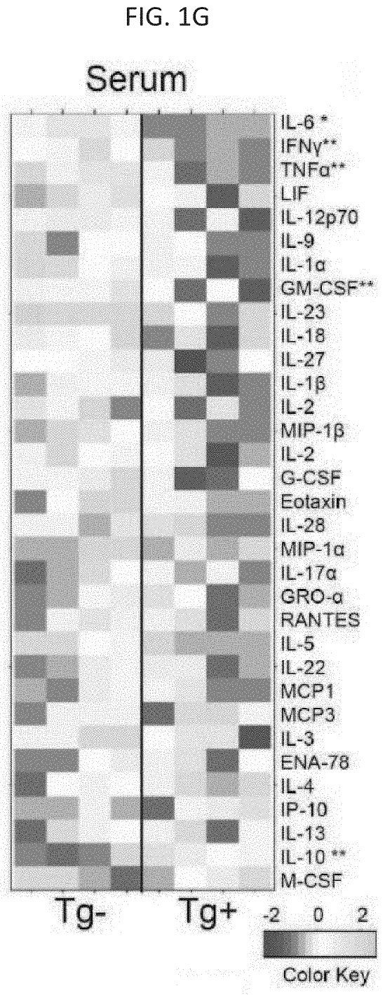

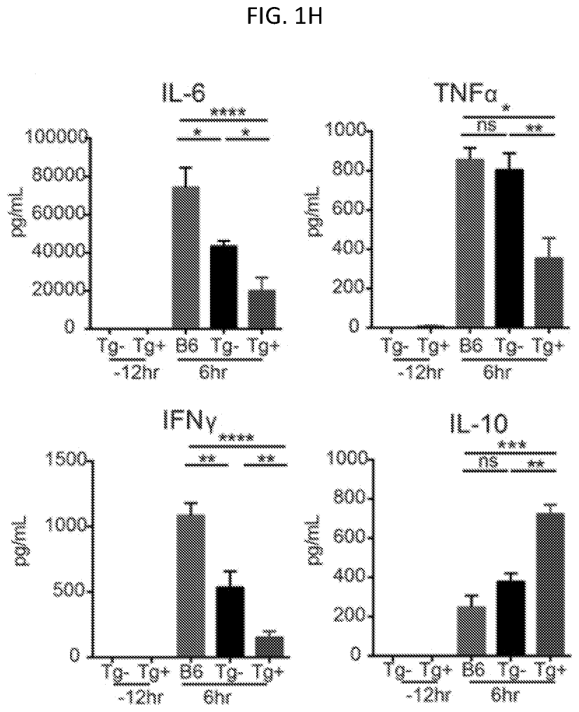

Experiments disclosed herein use transgenic mice that express hRetn (hRETNTg+) to study the function of hRetn in a mouse model of sepsis. LPS injection resulted in significantly increased circulating hRetn in the hRETNTg+ mice in FIG. 1B, which were critically protected against fatal LPS-induced inflammation compared to littermate control hRETNTg− mice in FIG. 1C. Further, therapeutic treatment with recombinant hRetn protected C57BL/6 mice against LPS-induced mortality in FIG. 2B. The role of hRetn to helminth-induced immunomodulation was tested, and it was observed that hRetn enhanced the protective effects of Nippostrongylus brasiliensis (Nb) infection in LPS-induced endotoxic shock in FIG. 3A. Mechanistically, hRetn inhibited LPS-induced neutrophilia in FIG. 2F, and promoted a shift from a pro-inflammatory signaling (e.g., TNFα, NF-κB) to an anti-inflammatory pathway (e.g., IL-10, STAT3) in FIG. 3E. Combining protein modeling, hRetn N-peptide synthesis, and immunoprecipitation assays, direct evidence is shown, for example, in FIGS. 4B, 4G, and 5I that hRetn binds TLR4 through the N-terminal helix and competes for the binding of the co-receptor MD2. In functional assays with human peripheral blood mononuclear cells (PBMC), hRetn binds to TLR4 and prevents subsequent LPS binding and inflammatory function through a STAT3 and TBK1-dependent mechanism in FIG. 6C. To test the effect of hRetn on TLR4 signaling in vivo, hRETNTg+ mice were generated in the Tlr4−/− background, and observed that the anti-inflammatory effects of hRetn were TLR4-dependent in FIG. 6A. Together, the present disclosure identifies a previously unrecognized role for hRetn in blocking LPS function and promoting anti-inflammatory pathways with important clinical implications for helminth-induced immunomodulation and sepsis.

### Example 2

hRETNTg⁺ mice are resistant to LPS-induced inflammation and mortality. Previous data suggest that hRetn is pathogenic in LPS-induced inflammation and sepsis; however, functional studies testing hRetn effects on sepsis pathogenesis have not been performed. To investigate this in vivo, transgenic mice that express hRetn (hRETNTg⁺) were used in a mouse model of LPS-induced septic shock (FIG. 1A). hRETNTg⁺ mice were previously generated by bacterial artificial chromosome-mediated integration of the hRETN gene and regulatory region on mouse resistin (mRetn−/−) background. Characterization of these mice revealed that circulating hRetn levels are comparable to humans, and that hRetn is significantly upregulated in vivo following LPS injection. In this disclosure, mice were challenged intraperitoneally (i.p.) with a low dose of LPS to induce hRetn expression in hRETNTg+ mice, followed by a second fatal dose of LPS. Low dose LPS led to significantly increased circulating hRetn in the hRETNTg+ mice (FIG. 1B). Strikingly, hRetn expression was protective against the fatal LPS dose, with significantly improved survival of hRETNTg+ mice compared to littermate control hRETNTg− mice (FIG. 1C, blue vs black). Although this two-dose LPS model may result in some endotoxin tolerance, this tolerance was not sufficient to protect against fatal endotoxic shock in the absence of hRetn. Subsequently the physiological and immune mechanism by which hRetn protected against LPS-induced mortality were investigated. Compared to hRETNTg⁻ mice, hRETNTg⁺ mice were protected from LPS-induced hypothermia (FIG. 1D). Flow cytometric analysis of the peritoneal exudate cells (PECs) from naïve mice revealed equivalent frequencies of macrophages, neutrophils and monocytes, but increased eosinophils in hRETNTg⁺ mice compared to hRETNTg⁻ mice (FIG. 1E). Following LPS treatment, the protective response in hRETNTg⁺ mice coincided with significantly reduced neutrophils and increased eosinophils compared to hRETNTg− mice, suggesting a shift from a pro-inflammatory response to a T helper type 2 (Th2) immune response. A significant increase in monocyte frequency was also observed. To identify cytokines which may contribute to hRetn-mediated protection against sepsis, the serum of LPS-treated hRETNTg⁻ or hRETNTg⁺ mice was analyzed with a luminex panel of 33 cytokines (FIG. 1F). LPS-treated hRETNTg⁺ mice exhibited a decrease in circulating pro-inflammatory and Th1 cytokines (e.g., TNFα, Interferon gamma (IFNγ), IL-6, IL-12, IL-1α and Granulocyte-macrophage colony-stimulating factor (GM-CSF)) compared to hRETNTg− mice (FIGS. 1G-1H). Conversely, the anti-inflammatory cytokine IL-10 was increased in hRETNTg⁺ mice.

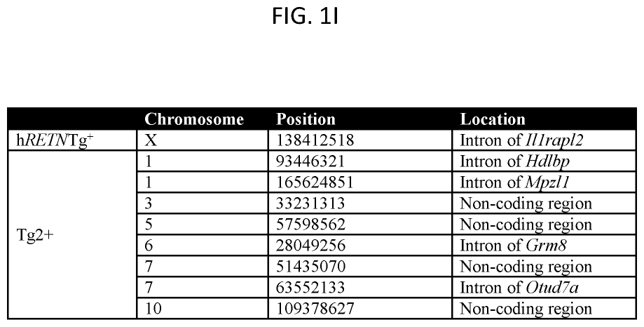

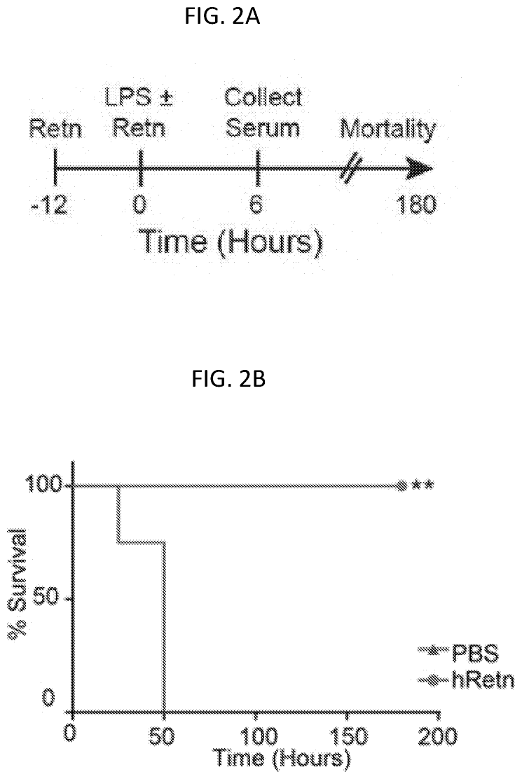

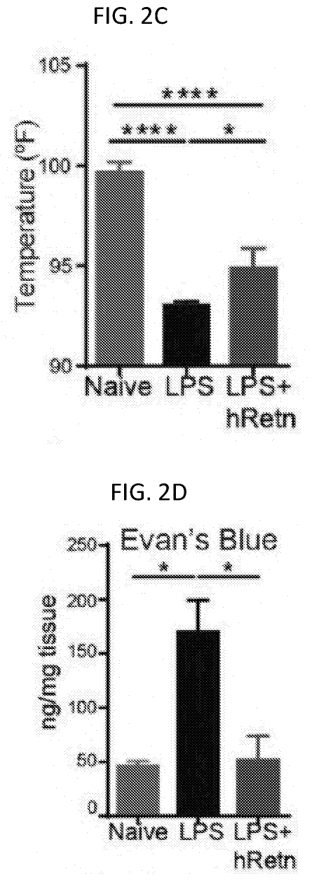

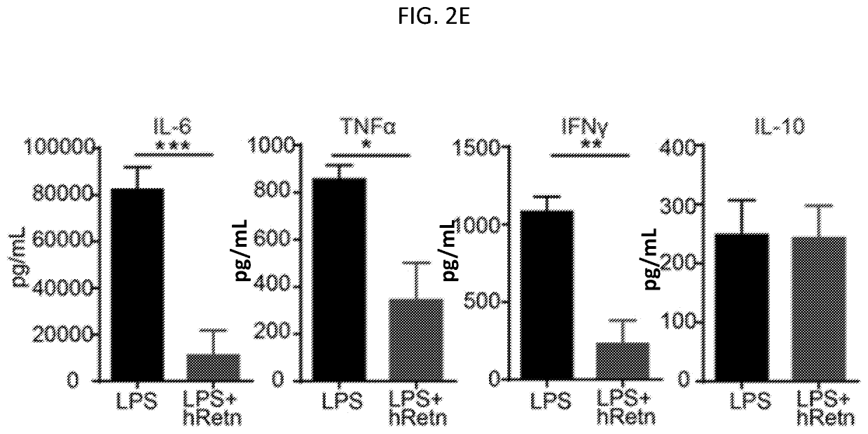

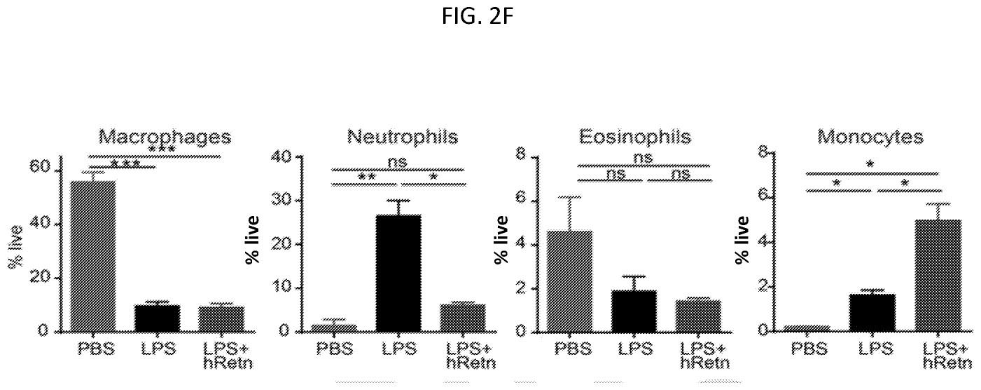

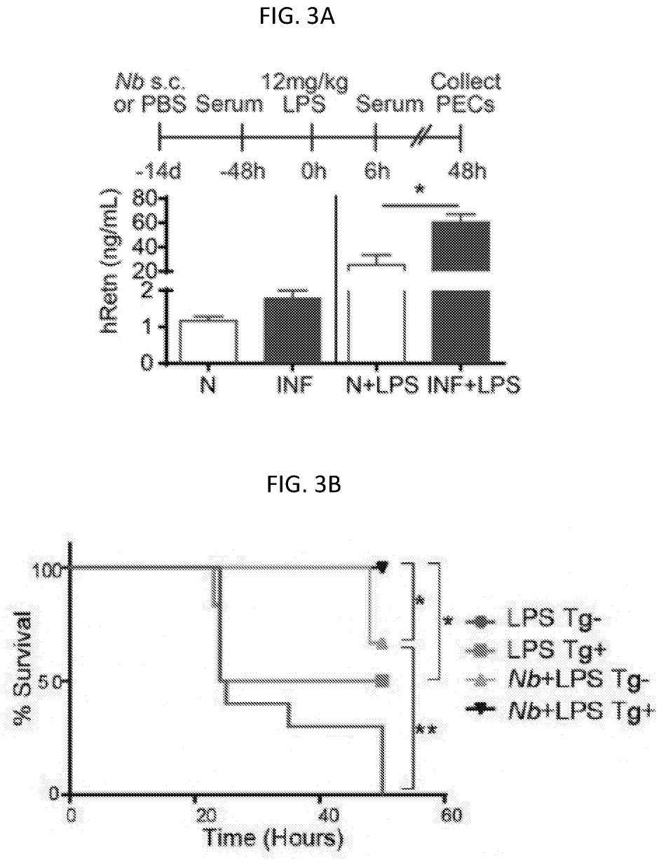

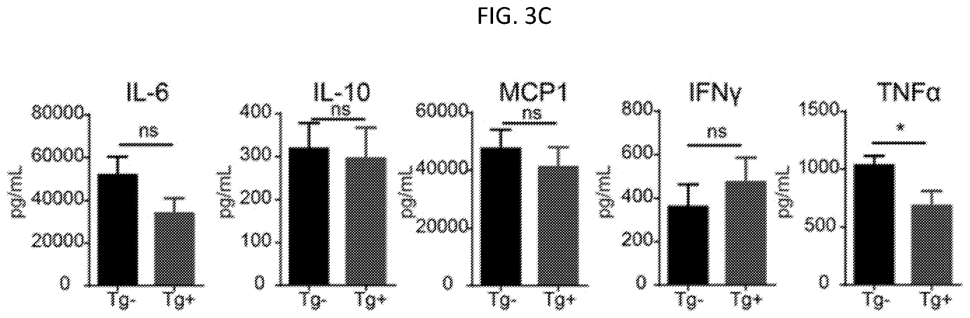

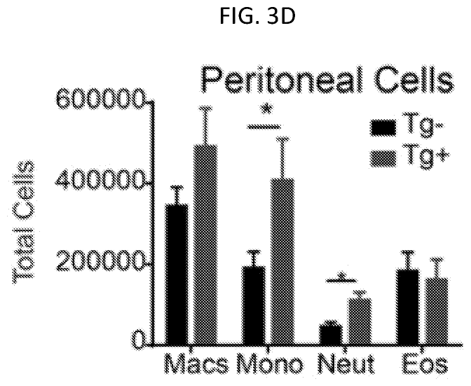

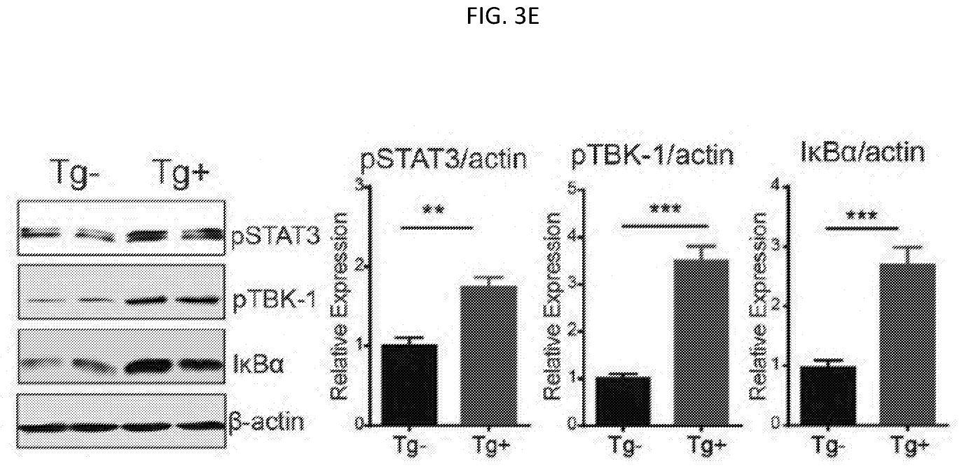

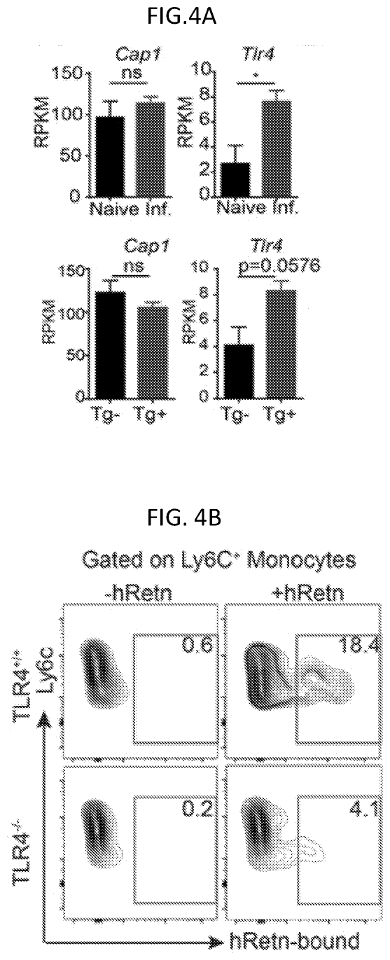

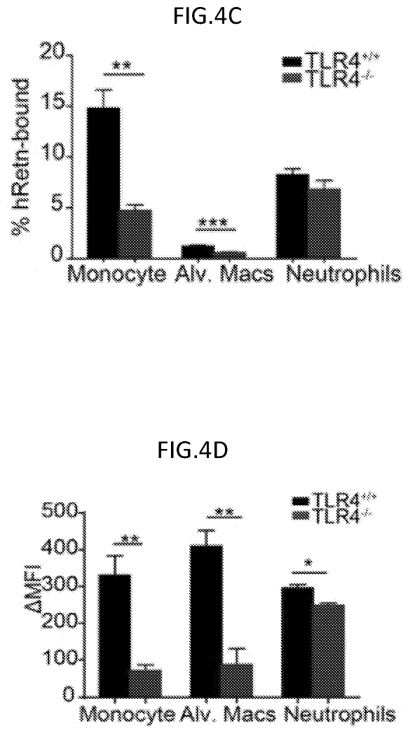

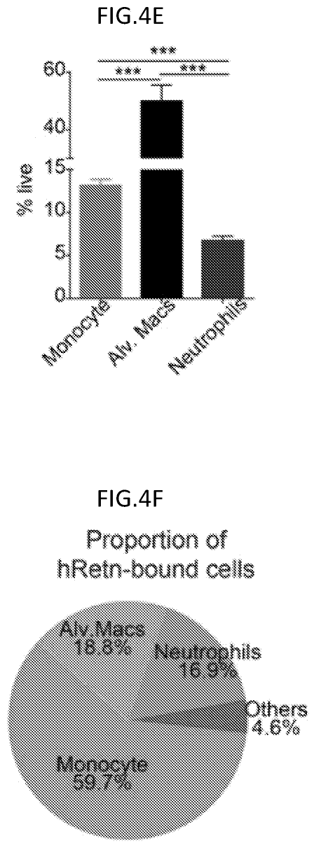

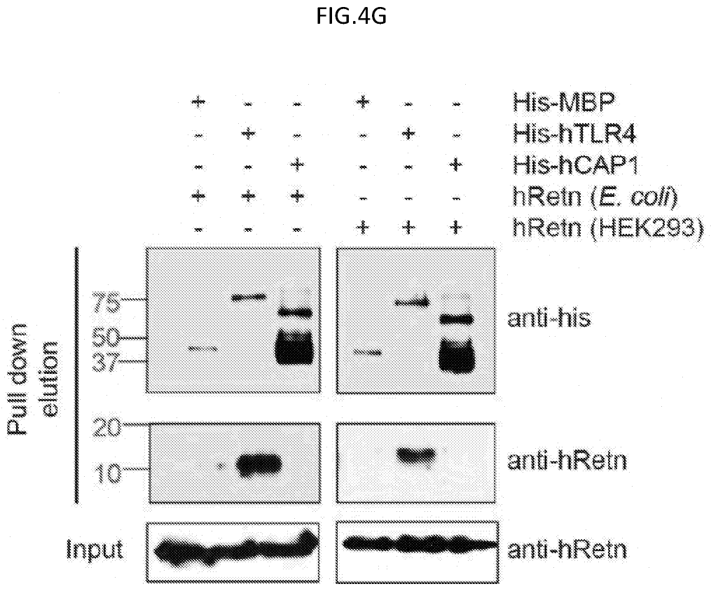

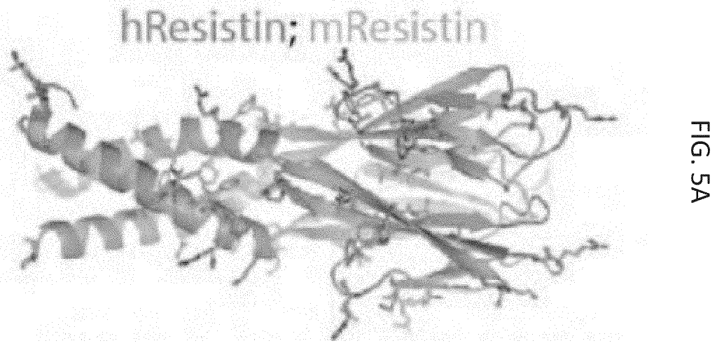

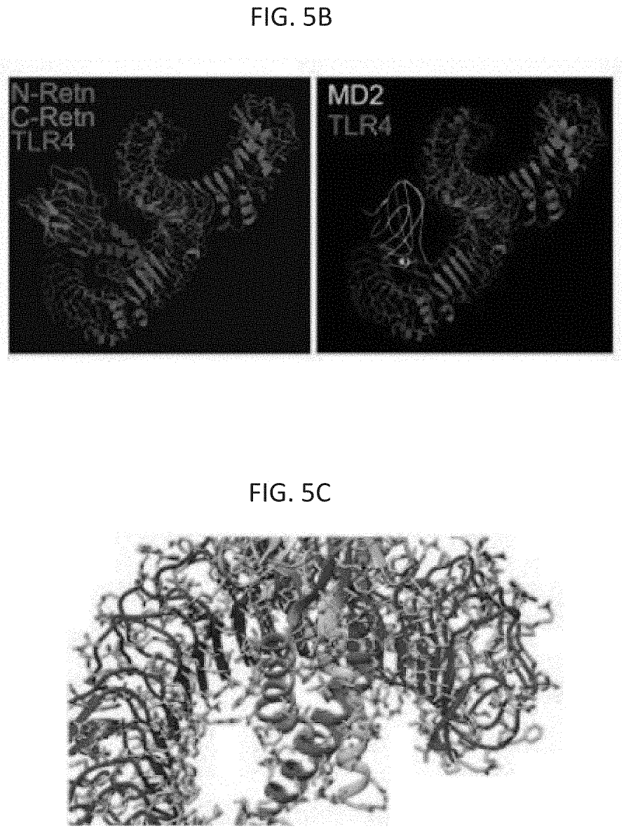

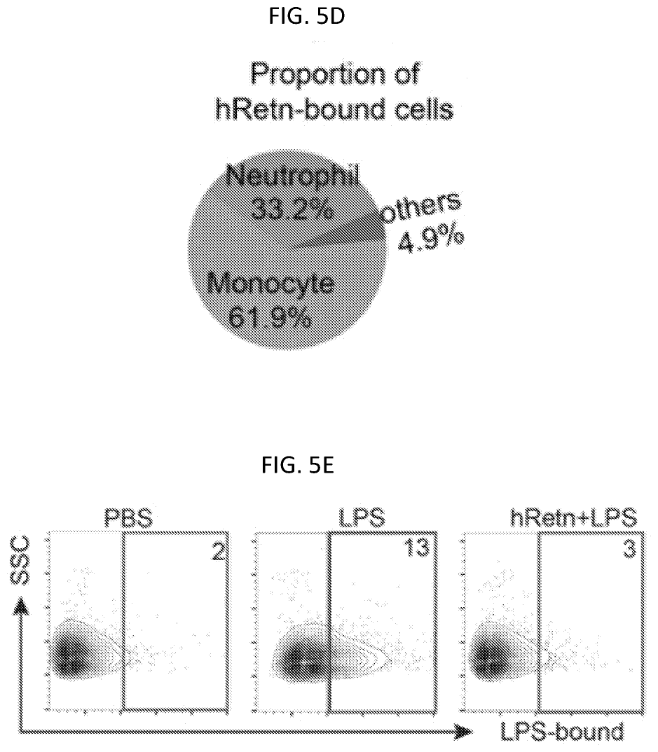

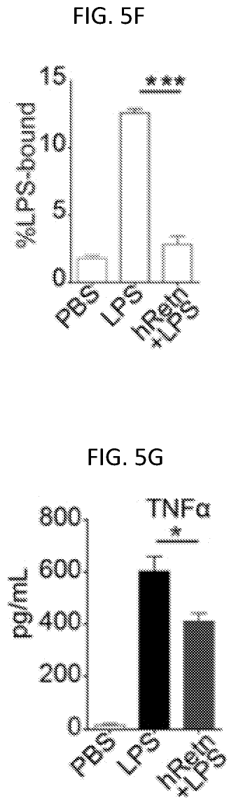

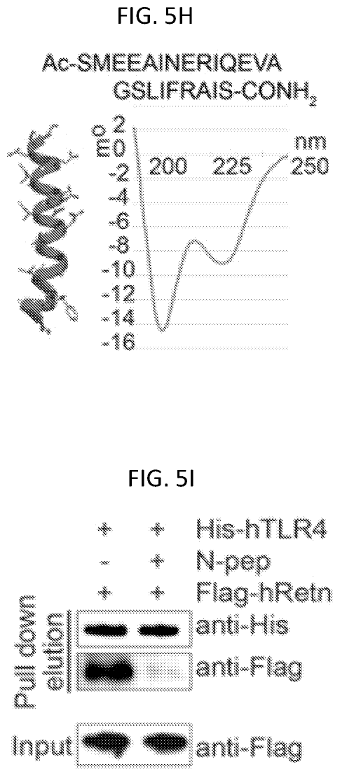

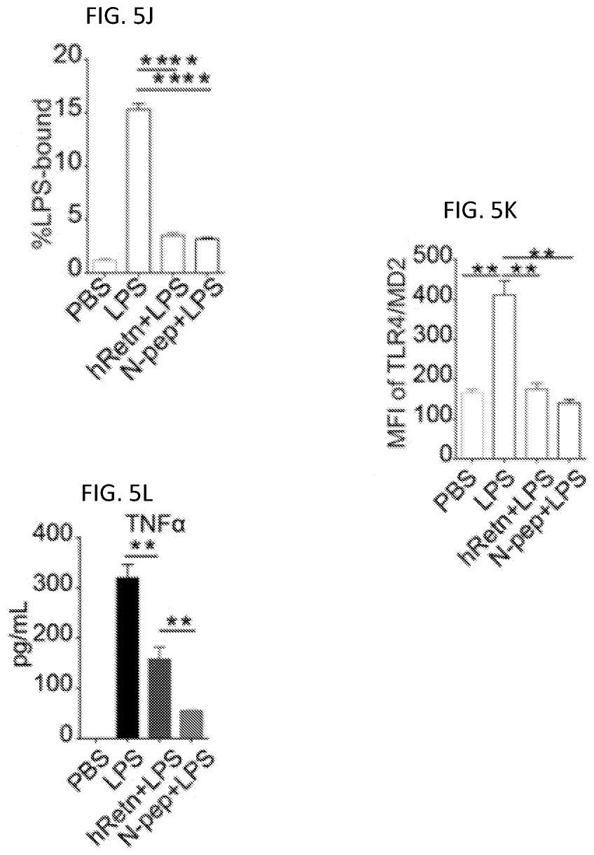

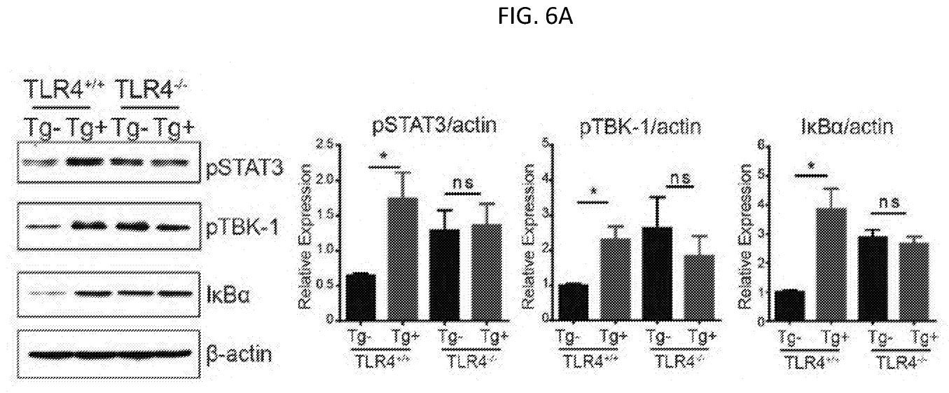

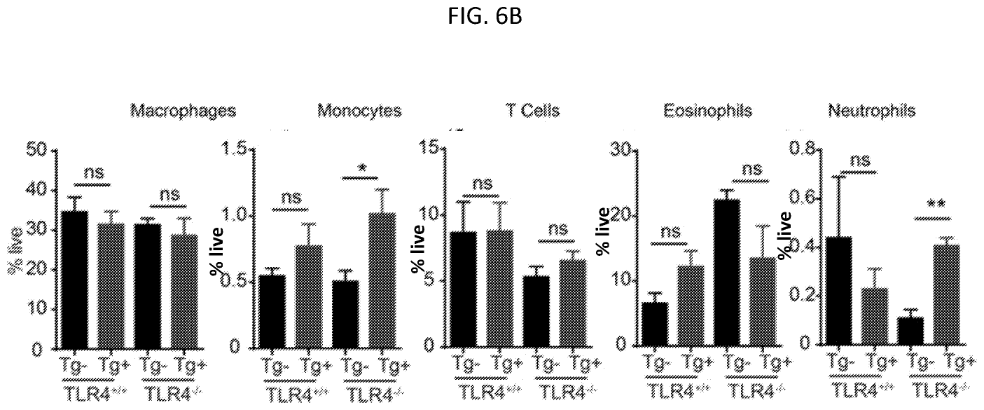

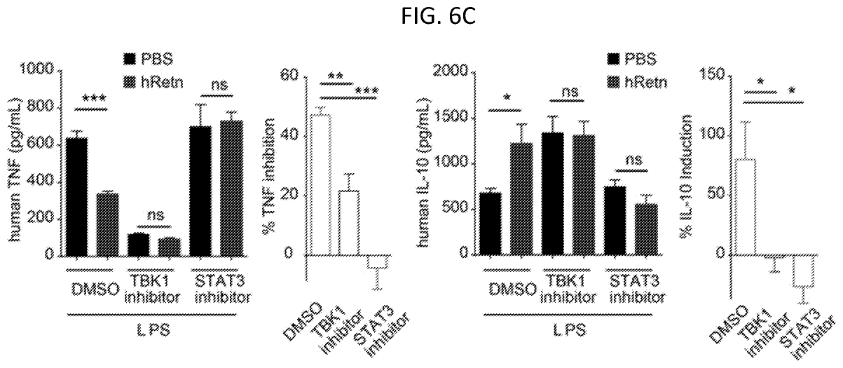

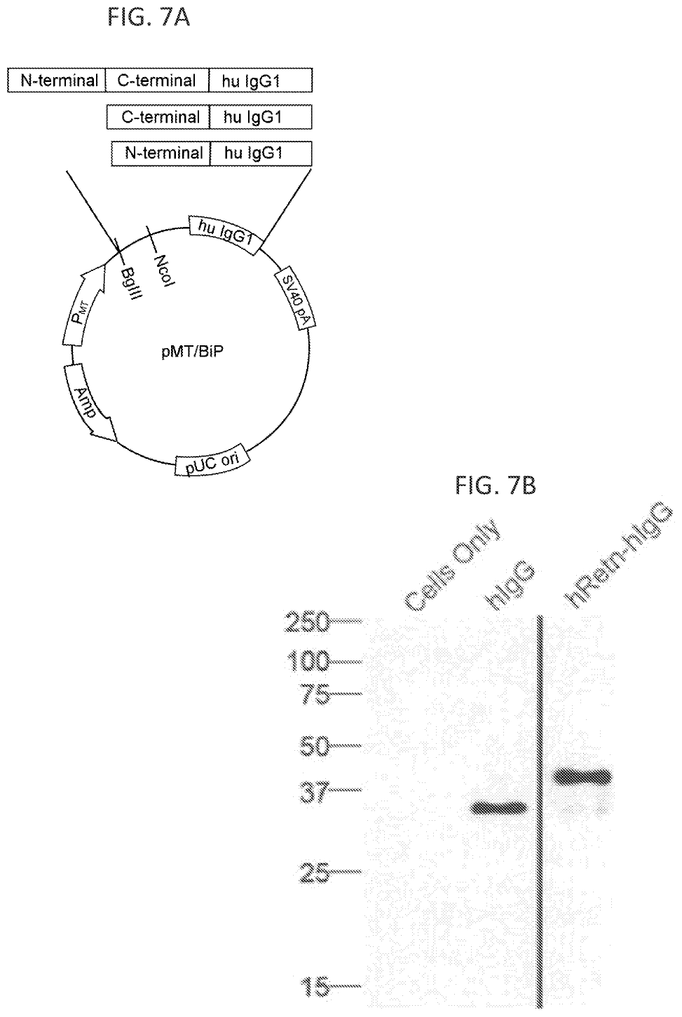

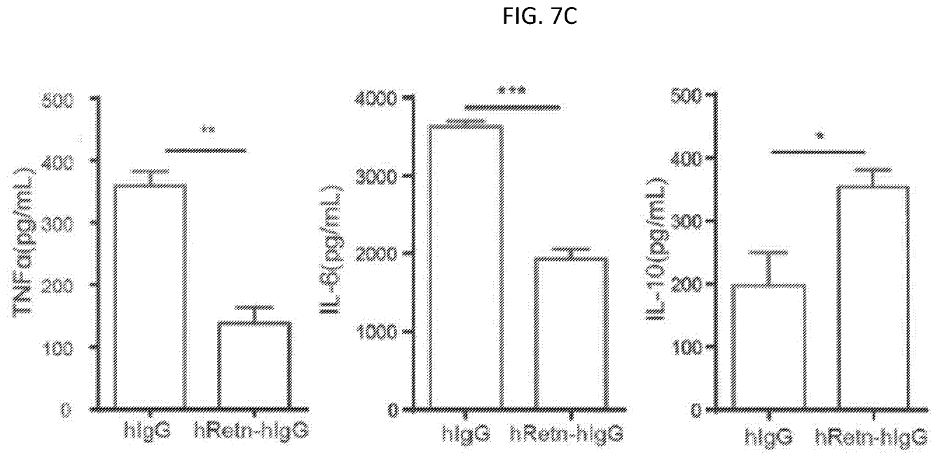

Given that the hRETN transgene was randomly integrated into the mouse genome, the protective effects observed in the transgenic mice could be due to disruption of another gene. Accordingly, a second transgenic mouse line (Tg2) was generated, and confirmed high circulating hRetn levels following LPS treatment (FIG. 1B). Compared to hRETNTg⁻ mice, Tg2 mice were also protected against the fatal LPS dose, exhibiting 100% survival (FIG. 1C, green). A single nucleotide polymorphism analysis was also used (from Dartmouse™), to locate the hRETN gene insertion sites in the transgenic mice at a resolution of 0.5 Mbp. In both transgenic mouse lines, hRETN gene insertions were predicted in non-coding regions or intron sites (FIG. 11). Since the hRETN transgene insertion did not disrupt coding regions, and there is no overlap between insert locations in the hRETNTg⁺ and Tg2 mice, it is unlikely that the protection conferred by the hRETN transgene in both mouse lines is an artifact of the transgene insertion. Both hRETN transgenic mouse lines were generated on a mRetn^{-/-} background, therefore, C57BL/6 mice were included in the LPS-induced septic shock model to account for potential effects of endogenous mRetn. In contrast to hRETNTg⁺ mice, C57BL/6 mice succumbed to the fatal LPS dose and mortality was correlated with increased circulating inflammatory cytokines (IL-6, TNFα and IFNγ) and decreased IL-10. Notably, the C57BL/6 mice had increased serum IL-6 and IFNγ than hRETNTg⁺ mice, suggesting that endogenous mRetn may increase these cytokines. However, this potential functional effect of mRetn did not affect survival outcome, with no significant differences in survival rate between hRETNTg− and C57BL/6 mice.

Together, these data identify an anti-inflammatory role for hRetn in endotoxic shock. Since neutrophils contribute to sepsis progression through the production of reactive oxygen species and pro-inflammatory cytokines the reduction of neutrophils and pro-inflammatory cytokines in hRetn-treated mice likely contributes to the reduced mortality during endotoxic shock. See, e.g., Kovach M A et al., 2012, “The function of neutrophils in sepsis,” Curr Opin Infect Dis 25(3):321-327, the entire content of which is herein incorporated by reference. Also, monocytes, potentially induced by the increased macrophage colony-stimulating factor (M-CSF) in the hRETNTg⁺ mice may have contributed to the increase in IL-10. The present disclosure shows a protective function for hRetn in reducing fatal LPS-induced mortality and challenges the current paradigm that resistin is an inflammatory cytokine that exacerbates sepsis pathogenesis.

### Example 3

Therapeutic administration of hRetn ameliorates LPS-induced inflammation and mortality. As a complementary approach to the hRETNTg⁺ mice, intraperitoneal treatment with recombinant hRetn was shown to be protective against endotoxic shock (FIG. 2A). Since recombinant hRetn in C57BL/6 mice were used, a preliminary dose of LPS was not necessary to induce hRetn expression and limited confounding factors caused by potential endotoxin tolerance. Compared to control C57BL/6 mice, which succumbed to LPS-induced sepsis, mice treated with recombinant hRetn were resistant to LPS-induced mortality (FIG. 2B). hRetn-mediated effects were associated with a modest protection from the LPS-induced temperature drop, and significantly reduced LPS-induced vascular permeability (FIGS. 2C and 2D). Associated with hRetn mediated protection from sepsis pathogenesis, a significant reduction in pro-inflammatory cytokines TNFα, IL-6 and IFNγ was observed, with no change in IL-10 expression (FIG. 2E). Similar to the hRETNTg⁺ mice, hRetn-treated mice exhibited significantly reduced LPS-induced neutrophils in the peritoneal cavity and increased monocytes (FIG. 2F). The recombinant hRetn used was generated in bacteria, but had undetectable endotoxin (≤0.016 U/ug) when quantified by the limulus amebocyte lysate (LAL) assay. Together, these results demonstrate that both transgenic expression of hRETN and hRetn treatment are critically protective in a mouse model of sepsis by limiting pro-inflammatory cytokine expression. Treatment with hRetn did not entirely recapitulate the phenotype in hRETNTg⁺ mice, notably, the differences in eosinophils and IL-10 expression. These differences are possibly due to effects of timing of expression, or half-life or localization of the recombinant hRetn. Nonetheless, the protective effect of exogenous hRetn treatment supports the therapeutic potential of hRetn in altering the outcome of septic shock.

### Example 4

Helminth infection-induced hRetn protects against sepsis. Helminth infections are associated with an increase in circulating LPS, presumably due to organ damage or an increase in intestinal barrier permeability as described in Farid et al., 2008, “Increased intestinal endotoxin absorption during enteric nematode but not protozoal infections through a mast cell-mediated mechanism,” Shock 29(6):709-716, the entire contents of which are herein incorporated by reference. Additionally, Nippostrongylus brasiliensis (Nb)-infected hRETNTg⁺ mice were reported to have significantly elevated hRetn in the infected tissue, which impaired optimal helminth expulsion. It was hypothesized that instead of promoting anti-helminth immunity, hRetn may limit bacterial or LPS-induced inflammatory responses. To investigate this possibility, naïve or day 14 Nb-infected hRETNTg⁺ and hRETNTg⁻ mice were injected with a fatal dose of LPS and monitored for symptoms of septic shock for 48 hours. As opposed to the previous models of endotoxic shock where hRetn expression or treatment occur 12 hours prior to fatal LPS challenge, this model investigated the effect of Nb-induced hRetn over 14 days. Circulating hRetn was modestly increased in Nb-infected mice, but significantly increased after LPS challenge compared to naïve mice, (FIG. 3A). While all naive hRETNTg⁻ mice succumbed to the fatal LPS dose, naive hRETNTg⁺ mice were more resistant to endotoxic shock with 50% survival, suggesting that homeostatic hRetn levels are protective (FIG. 3B). Nb infection conferred partial protection to hRETNTg− mice; however, this protective effect was significantly enhanced by hRetn (100% survival of Nb-infected hRETNTg⁺ mice). Cytokine quantification of the serum from Nb-infected mice revealed equivalent levels of LPS-induced monocyte chemoattractant protein 1 (MCP1), IFNγ, IL-6 and IL-10 (FIG. 3C). This was in contrast to the low and high dose LPS challenge where there was significantly reduced IFNγ and conversely increased IL-10. Given that mice were infected with Nb 14 days prior to LPS challenge, it is possible that Nb-induced IL-10 in the hRETNTg⁺ mice may have occurred earlier, or that in this chronic situation, the effect of hRetn in reducing TNFγ is more significant than its effect in increasing IL-10.

Flow cytometric analysis of the peritoneal cavity of Nb-infected revealed significantly increased neutrophils and monocytes in the hRETNTg⁺ mice (FIG. 3D). These data suggest that while hRetn induced by low dose LPS and Nb infection both protect from septic shock, the underlying immune mechanism of protection may be different. The increased infiltration of neutrophils in Nb-infected hRETNTg⁺ mice suggests that neutrophils are not the direct cause of hRetn-mediated protection in this context and may not be inflammatory in helminth infection. The next experiments investigated if Nb-induced hRetn influenced pro and anti-inflammatory signaling following LPS injection. Peritoneal cells from Nb+LPS treated hRETNTg⁻ and hRETNTg⁺ mice were flash frozen, lysed and analyzed by Western blot. These experiments showed that TLR4-mediated anti-inflammatory signaling pathways were induced in hRETNTg+ mice (FIG. 3E). In particular, phosphorylation of TBK1 was increased and NF-κB inhibitor, alpha (IκBα) protein degradation was decreased. Additionally, there was elevated levels of phosphorylated STAT3, suggesting increased IL-10 signaling. Together, these data suggest that Nb-induced hRetn expression protects from endotoxic shock by promoting anti-inflammatory signaling pathways.

### Example 5

Human resistin binds to Toll-like Receptor 4 (TLR4). To identify the downstream mechanism by which hRetn inhibits LPS-induced inflammation, experiments were designed to investigate the contribution of putative hRetn receptors. There are currently two proposed receptors for hRetn: TLR4 and cyclic adenylate associated protein 1 (CAP1). Experiments in this disclosure investigated RNA-seq datasets from naïve or Nb-infected lungs of hRETNTg⁻ and hRETNTg⁺ mice for differential expression of Cap1 and Tlr4. Although Cap1 expression was not influenced by Nb infection, Tlr4 expression was significantly induced by Nb infection (FIG. 4A). Notably only Tlr4 expression was increased in hRETNTg+ mice (FIG. 4A), suggesting that hRetn may promote expression of its own receptor. Subsequent experiments utilized the hRetn cellular binding assay to test if hRetn binding was through TLR4. Dissociated lung cells from Nb-infected Tlr4+/+ or Tlr4−/− mice were incubated with hRetn followed by capture with detection antibodies to hRetn. While Tlr4+/+ monocytes (Ly6C+CD11b+) were able to bind hRetn, hRetn binding was significantly abrogated in Tlr4−/− monocytes (FIG. 4B). The TLR4-dependent binding was quantified as a percentage hRetn-bound cells and mean fluorescence intensity (MFI) in monocytes, alveolar macrophages (F4/80+CD11c+) and neutrophils (Ly6G+CD11b+) (FIGS. 4C and D). TLR4-dependent binding in monocytes and alveolar macrophages was observed, but not neutrophils where hRetn binding in the absence of TLR4 was not noticeably affected. While TLR4 is a receptor for hRetn there are alternate hRetn receptors, which may be selectively expressed by different cell-types. Thus, neutrophils could preferentially express an unidentified receptor for hRetn, which binds to and modulates the pro-inflammatory response through an alternative pathway. As such, the relative contribution of each hRetn-bound immune cell population was investigated by measuring their frequency in the lung (FIG. 4E). By this measurement, monocytes were the dominant cell-type that bound resistin, followed by neutrophils and alveolar macrophages (FIG. 4F). Although the frequency of hRetn-bound cells was relatively low (15% in monocytes and 8% in neutrophils), this low frequency of hRetn responsive cells was nevertheless sufficient to prevent fatal endotoxic shock. Staining for B cells, T cells, and eosinophils, however, we did not observe any other significant population that bound hRetn.

Given that there are no publicly available CAP1 deficient mice, a direct protein-protein interaction of hRetn to TLR4 or CAP1 was investigated as an alternative to cell binding assays. A pull-down assay was performed utilizing recombinant hRetn with histidine (his)-tagged human TLR4, his-human CAP1 or his-maltose binding protein (MBP) as a negative control (FIG. 4G). In the pull-down elution, Western blot using anti-his antibody confirmed that his-MBP, his-TLR4 and his-CAP1 are all pulled down by the nickel-NTA agarose beads. In addition, when blotting the elution fraction with anti-hRetn antibody, hRetn was only present in the elution with his-TLR4, not his-CAP1 or his-MBP. To ensure that this interaction was not due to low-level LPS contamination, this pull-down assay was also performed with mammalian cell-derived hRetn. Similar to E. coli-derived hRetn, mammalian-derived hRetn is only detected when pulled down with his-TLR4, not his-CAP1 or his-MBP. Unlike previous studies, where immunoprecipitation was performed with cell lysates, this data provides direct evidence that hRetn binds to TLR4 and does not require other proteins to form a complex. While hRetn does not directly bind to CAP1, this data does not exclude the possibility that hRetn might bind to CAP1 if other adaptor proteins are present.

### Example 6

hRetn competes with LPS/MD2 for binding to TLR4. Although the X-ray crystal structure of hRetn is not available, the crystal structure of mouse Retn (mRetn) and human TLR4 were used to model hRetn binding to TLR4. With reference to FIG. 5A, this revealed that hRetn (green) and mRetn (cyan) have the same basic structure; a trimer consisting of a triple-helix stem (N-terminal domain) and a jelly-roll like head (C-terminal domain). Next, the ClusPro program was used to predict the interactions between hRetn and TLR4 as described in Kozakov D, et al. (2017) The ClusPro web server for protein-protein docking. Nat Protoc 12(2):255-278, the entire content of which is herein incorporated by reference. As a first step, MD2, the adaptor protein that mediates LPS binding to TLR4 was docked, and several possible solutions proposed by ClusPro were found that closely match the solved crystal structure of the complex (FIG. 5B). Subsequently, the model of hRetn into human TLR4 was docked and several solutions place the N-terminal of hRetn hexamer (blue) within the binding pocket of TLR4 (red) for MD2 (white). In various predicted docked models obtained with ClusPro, the junction between the stem and head fits into the inner face of the horseshoe-like TLR4 molecule, obstructing the binding domain for MD2 and LPS. In these poses, several Retn side-chains interact with different regions of the TLR4 to make ionic interactions and hydrogen bonds (FIG. 5C). Based on these structural predictions, it was hypothesized that hRetn may sterically block LPS/MD-2 from binding to TLR4 thereby inhibiting LPS-induced inflammation. To investigate this possibility, in vitro competitive resistin/LPS binding assays were performed on human PBMCs. Evaluation of hRetn binding in human PBMC revealed that CD11b+CD14+ monocytes were the dominant population that bound hRetn, followed by SSChiCD11b+CD16+ neutrophils (FIG. 5D). Therefore, the potential for hRetn to block LPS binding in monocytes was assessed. With reference to FIGS. 5E-5F, monocytes were able to bind LPS, however, binding was significantly abrogated if cells are pre-incubated with hRetn. To determine whether hRetn inhibited downstream LPS function, human PBMCs that had been pre-incubated with PBS or hRetn followed by LPS stimulation were examined. PBMCs treated with only LPS generated significantly more TNFα than cells treated with hRetn and LPS, demonstrating a functional inhibition of LPS-induced inflammation (FIG. 5G). These results were not an effect of endotoxin contamination, as the recombinant hRetn used in these studies was derived from HEK293 cells. Additionally, since LPS can activate both TLR4 and TLR2, ultrapure LPS derived from Salmonella Minnesota was used, which binds to and activates TLR4 and does not bind to TLR2.

Experiments were subsequently designed to test the accuracy of the hRetn/TLR4 modeling, which predicted binding of the hRetn N-terminal helix to hTLR4. To this end, a solid phase synthesizer was used to generate an hRetn N-peptide (1-23 a.a.) (SEQ ID NO: 2) and test its helical structure and function. The N-terminal sequence of hRetn contains many amino acids known to have a high propensity to form and stabilize alpha helices. For example, the presence of an RxxE motif, which may allow the two amino acids side-chains to form an intramolecular salt bridge and would help to stabilize the alpha helix in both the full-length form and the synthesized peptide. Circular dichroism (CD) analysis revealed that the synthetic agent has a significant alpha-helical content in solution, with negative bands around 208 nm and 222 nm and a positive band at 190 (FIG. 5H). Given the small size of the hRetn N-peptide and lack of specific antibodies to the N-peptide, it was not possible to perform the hTLR4 pulldown assay with the N-peptide. Instead, experiments were designed to test the ability of the hRetn N-peptide to competitively inhibit subsequent hRetn binding, using the anti-Flag antibody to detect Flag-tagged full-length hRetn. Pre-incubation of hTLR4 with the hRetn N-peptide was sufficient to abrogate binding of full-length hRetn binding (FIG. 5I).

With reference to FIG. 5J, the functional ability of the hRetn N-peptide to block LPS binding and function was tested. The binding experiments were performed on ice to rule out effects of LPS-induced TLR4 endocytosis, which would have added complexity for interpretation of the data. Pre-incubation of human PBMC with the hRetn N-peptide or full-length hRetn inhibited LPS binding to CD14+CD11 b+ monocytes. As shown in FIG. 5K, flow cytometric analysis surface MD2/TLR4 revealed that LPS treatment promoted MD2/TLR4 complex formation, but this was abrogated by hRetn or hRetn N-peptide. Functionally, the hRetn N-peptide was significantly more efficient at suppressing LPS-induced TNFα than full-length hRetn (FIG. 5L). An additional formulation of human resistin fused to the humanFc Ig fragment was also effective at inhibiting proinflammatory cytokines and promoting IL-10 expression in primary macrophage cultures (FIG. 7C). These data suggest that hRetn binds TLR4 through its N-terminal helix and effectively inhibits LPS binding and pro-inflammatory function. Together, these studies support the findings of the hRETNTg⁺ mouse model, and validate that hRetn also inhibits LPS responsiveness in human immune cells.

### Example 7

hRetn regulates anti-inflammatory signaling pathways through TLR4. To investigate whether hRetn signals through TLR4, hRETNTg⁺ Tlr4−/− mice were generated on a mRetn−/− background. Since Tlr4−/− mice are resistant to endotoxic shock, experiments were designed to investigate whether hRetn had TLR4-dependent anti-inflammatory effects under homeostatic conditions. Western blot for anti-inflammatory signaling molecules was performed on unstimulated peritoneal cells recovered directly from hRETNTg⁻ or hRETNTg⁺ mice on the Tlr4+/+ or Tlr4−/− background. Consistent with an anti-inflammatory function for hRetn, peritoneal cells from hRETNTg⁺ Tlr4+/+ mice had increased pSTAT3, pTBK-1 and decreased IκBa degradation compared to hRETNTg-Tlr4+/+ mice as shown in FIG. 6A. However, this anti-inflammatory effect was abrogated in the absence of TLR4, where there was no significant difference between hRETNTg+Tlr4−/− and hRETNTg-Tlr4−/− mice. With reference to FIG. 6B, peritoneal cells from naïve mice were also characterized by flow cytometry, which revealed modest increases in monocytes and neutrophil in hRETNTg⁺ compared to hRETNTg⁻ mice on the Tlr4−/− background but no significant differences in other cell populations, and on the Tlr4+/+ background.

With reference to FIG. 6C, experiments designed to determine whether hRetn effects on TLR4 signaling were dependent on the TBK1 and STAT3, human PBMC functional assays with TBK1 and STAT3 pharmacologic inhibitors were conducted. hRetn pre-treatment with control DMSO significantly reduced LPS-induced TNFα secretion and conversely increased IL-10 expression. However, treatment with TBK1 inhibitor prior to PBS or hRetn addition resulted in significantly reduced LPS-induced TNFα and conversely increased IL-10 expression. This suggests that LPS-induced TBK1 signaling is inflammatory in human PBMC. Given that TBK1 inhibitor treatment itself caused a similar anti-inflammatory effect to hRetn, it is difficult to definitively conclude whether hRetn functions through TBK1. Nonetheless, hRetn's functional effect was abrogated in the presence of TBK1 inhibitor, suggesting that hRetn cannot further downregulate TNFα or upregulate IL-10 in the absence of TBK1 signaling. hRetn's functional effect was also abrogated when STAT3 signaling was inhibited. Combined, these in vivo and in vitro data suggest that under homeostatic conditions, hRetn binds to TLR4 and promotes STAT3 and TBK1 signaling to prevent LPS pro-inflammatory effects.

### Example 7

One main pathogenic feature of sepsis is excessive inflammatory cytokine production, known as the systemic inflammatory response syndrome (SIRS), which contributes to septic shock and mortality. According to experiments and embodiments of the present disclosure, a novel mechanism has been identified for hRetn protecting against endotoxic shock by blocking LPS-TLR4 interaction and excessive production of pro-inflammatory cytokines. According to the present disclosure, two hRetn-expressing transgenic mouse lines, exogenous recombinant hRetn treatment, and human PBMCs cultures, show that hRetn is critically protective against fatal LPS-induced endotoxic shock.

Aspects of examples and embodiments of the present disclosure confirm that hRetn expression is increased during septic shock. However, while the present disclosure is not limited to any particular mechanism or theory, it is believed that rather than promoting inflammatory cytokines and to LPS-induced mortality, hRetn acts as a feedback mechanism to control systemic inflammation by binding and inhibiting TLR4 signaling. Although patients with more severe clinical scores for sepsis have more circulating hRetn, the data of the present disclosure suggests that the increased hRetn expression may be the body's attempt to limit the excessive inflammatory immune response.

The hRETNTg+Tlr4−/− mice were used to demonstrate that there are alternate receptors for hRetn. Interestingly, TLR4 deficiency abrogated the anti-inflammatory effect of hRetn in naïve mice, suggesting that hRetn's immunoregulatory effect is dependent on its functional interaction with TLR4. The potential role of CAP1 in hRetn-mediated inhibition of LPS function is unclear. The data of the present disclosure suggest that CAP1 is unlikely to directly mediate protection during endotoxic shock. The present disclosure presents direct hRetn-TLR4 interaction that functionally impacts LPS-induced signaling and function. Additionally, the present disclosure provides functional evidence that hRetn inhibits LPS binding to human immune cells and subsequent LPS inflammatory function through a STAT3 and TBK1 dependent mechanism. Through structural modeling and experimental studies with the N-terminal peptide of hRetn it was shown and concluded that hRetn interacts with the TLR4 monomer and inhibits binding of the MD2 adaptor protein. Competitive co-immunoprecipitation and human PBMC functional assays showed that the hRetn N-terminal is sufficient to bind to TLR4 and inhibit LPS-induced pro-inflammatory effects. These results support the hRetn N-terminal as the active domain of hRetn.

There is 60% sequence homology between mRetn and hRetn, however, mRetn expression is restricted to adipocytes while hRetn is predominantly expressed in myeloid cells. Given this caveat, transgenic hRETNTg⁺ mice on a mRetn−/− background were used where hRetn expression was validated by macrophages and monocytes. Both C57BL/6 (mRetn+/+) mice and mRetn−/− mice were equally susceptible to LPS-induced mortality, suggesting that endogenous mRetn does not inhibit LPS function. The differential expression pattern of mRetn and hRetn, may explain this dichotomy in function, whereby mRetn affects metabolic function and hRetn is expressed systemically where it has an immunoregulatory function.

Although neutrophils act to limit initial bacterial or viral infection, they can also contribute to sepsis through excessive production of pro-inflammatory cytokines. Examples and embodiments of the present disclosure show that hRetn inhibited neutrophil responses, associated with a decrease in the neutrophil chemoattractant GM-CSF, but that hRetn's effect on neutrophils may depend on the inflammatory context. In an LPS-alone model, hRetn reduced total neutrophil recruitment. In contrast, hRetn increased neutrophil numbers in Nb+LPS treated mice, but this increased neutrophilia was associated with protection from endotoxic shock. Recent studies have shown that helminth-induced neutrophils are significantly different from LPS-induced neutrophils and are not pro-inflammatory as described in Chen F, et al., 2014, “Neutrophils prime a long-lived effector macrophage phenotype that mediates accelerated helminth expulsion,” Nat Immunol 15(10):938-946, the entire content of which is herein incorporated by reference. Therefore it is likely that the hRetn-induced neutrophils in Nb-infected mice did not contribute to LPS-induced inflammation.

hRetn binding assays of mouse and human immune cells revealed that monocytes were the main cell-type that bound hRetn. Additionally, monocyte frequencies were increased in vivo in hRETNTg⁺ mice and recombinant hRetn-treated mice. Together, these data suggest that monocytes are the main downstream cellular target of hRetn, where hRetn acts to suppress inflammatory pathways while promoting anti-inflammatory signaling through binding TLR4. The data and embodiments of the present disclosure support an immunoregulatory function for hRetn through direct effects on monocytes.

Homeostatic levels of hRetn in the transgenic mice were only 50% protective against fatal endotoxic shock. Instead, low dose LPS treatment or Nb infection was required to boost circulating hRetn levels provided optimal protection against subsequent endotoxic shock. hRETNTg⁺ mice exhibited a TLR-4 dependent increase in TBK-1 signaling, but decrease in NF-κB signaling, supporting a model where hRetn binds to TLR4 and preferentially activates TRIF/TBK1. These results suggest that hRetn binding to TLR4 may decrease inflammation in two ways, by inhibiting LPS binding and proinflammatory signaling, while increasing TBK1 activity and IL-10 production.

While helminth infection is associated with an increase in circulating LPS, there are numerous helminth-mediated immunoregulatory mechanisms in place to reduce LPS-associated inflammatory response. The data of the present disclosure suggests that hookworm infection also protects from sepsis through hRetn dependent and independent mechanisms. Nb-infected hRETNTg⁻ mice were more resistant to LPS-induced mortality compared to naïve hRETNTg− mice. However, hRetn significantly enhanced Nb-induced protection from endotoxic shock. These data map hRetn as a helminth-induced regulatory pathway upstream of IL-10 expression. The complexity of host-helminth interaction is the result of millions of years of co-evolution. For a positive outcome, the infected host may effectively balance the immune response to limit damage caused by not only the pathogen, but also excessive inflammation. This balance is essential when the host is co-infected with a variety of pathogens, such as helminths and bacteria. The examples and embodiments of the present disclosure show that while hRetn exacerbates helminth burden, it protects the host from excessive inflammation caused by endotoxic shock by blocking interaction between LPS and TLR4. In turn, this mechanism is exploited by helminths to prevent their own expulsion.

### Example 8. Materials and Methods

Mice. Human resistin transgenic mice were generated on a mouse Retn−/− background as previously described in Park et al., 2011, “Inflammatory induction of human resistin causes insulin resistance in endotoxemic mice,” Diabetes 60(3):775-783, the entire content of which is herein incorporated by reference. Briefly, the human resistin gene, along with 21,300 bp upstream and 4,248 bp downstream of the human resistin start site were inserted through a bacterial artificial chromosome (BAC). Genome insertion of hRETN in the two transgenic mouse lines was determined by Dartmouse™, which sequences and analyzes thousands of SNPs throughout the mouse genome. For the endotoxic shock model, mice were injected i.p. with two doses of LPS (Sigma): 0.05 mg/kg LPS followed by 12 mg/kg LPS (females) or 20 mg/kg LPS (males) 12 hours later. Mice were monitored at least twice a day and euthanized according to humane endpoints. For treatment with recombinant resistin, C57BL/6 mice were injected i.p. with PBS or 0.5 μg hRetn (Peprotech). Nippostrongylus brasiliensis life cycle was maintained in Sprague-Dawley rats, as previously described in Jang et al., 2015, “Macrophage-derived human resistin is induced in multiple helminth infections and promotes inflammatory monocytes and increased parasite burden,” PLoS Pathog 11(1):e1004579, the entire content of which is herein incorporated by reference. Mice were anesthetized with isoflurane and injected subcutaneously with 500 L3 larvae. Serum collection was by retro-orbital bleeding and body temperature was measured by rectal thermometer (Braintree Scientific). All animals in the experiment were age-matched (6-8 week old), gender-matched and housed in a specific pathogen free facility.

Vascular Permeability Assay. Evan's blue dye was used to measure vascular permeability. Mice were anesthetized by isoflurane and 200 μL of 0.5% Evan's Blue dye in PBS was injected reto-orbitally. After 10 minutes, mice were euthanized by CO2 and perfused with 20 mL PBS. Tissue was excised, weighed and incubated with 500 μL N, N-dimethylformamide (Sigma) for 24 hours at 55° C. Extracted Evan's blue was measured at 610 nm according to a standard curve.

Binding Assay and Flow Cytometry. Single cell suspension of lung tissue was prepared and hRetn binding was measured as previously described in Jang et al., 2015, supra. Briefly, dissociated lung cells were incubated for 1 hour at 4° C. with 0.5 μg recombinant hResistin (Peprotech) or PBS, followed by 2× wash in FACS buffer, incubation with Fc block (5 μg/mL αCD16/32 and 10 μg/mL purified rat IgG1, 5 min at 4° C.). Cells were stained with biotinylated α-hRetn (Peprotech, 30 min at 4° C.) followed by detection with BV605-conjugated streptavidin (BD) and surface marker antibodies. To collect peritoneal exudate cells (PECs), the peritoneal cavity was washed and PECs recovered in 5 mL ice cold PBS. Surface marker antibodies were: F4/80 (clone A3-1), SiglecF (clone E50-2440), CD4 (clone RM4-5), Ly6C (HK1.4), CD11b (clone M1/70), CD11c (clone N418), Ly6G (clone 1A8), MHCII (clone M5/114.15.2) and CD3e (clone 145-2C11), CD115 (clone AFS98), purchased from Affymetrix, BD Biosciences or Biolegend. Cell populations were determined as follows: peritoneal macrophage (F4/80+CD11b+), neutrophils (Ly6G+CD11b+), eosinophils (SiglecF+CD11c−) and monocyte (Ly6C+CD11b+), alveolar macrophages (F4/80+CD11c+). For flow cytometric analysis, all samples were run on the BD LSRII and analyzed on FlowJo (v10).

Human PBMCs. Human buffy coat was purchased from Zen-bio. Buffy coat was overlaid on top of Histopaque-1077 and spun at 700g at 25° C. with no brake and PBMCs were recovered from the interphase. PBMCs were plated in 96-well plates and stimulated with mammalian-derived human resistin (1 μg/mL, LifeSpan Biosciences) or hRetn N-peptide (1 μg/mL). After 24 hours, ultrapure LPS (100 ng/mL, Invivogen) was added and supernatants recovered for ELISA at 24 hours. Where indicated, cells were incubated for 4 hours with STAT3 (5, 15-DPP, 50 μM) or TBK1 (BX-795, 1 μM) inhibitors prior to hRetn treatment. For the hRetn competitive binding assay, 1×106 cells were incubated with recombinant hRetn (0.5 μg), washed in PBS then incubated with 0.5 μg LPS-biotin (incubations were 30 minutes on ice). Cells were washed in FACS buffer, treated with Fc block, and stained with BV605-conjugated streptavidin and α-human primary antibodies: CD14 (clone M5E2), CD11 b (clone M1/70) and MD2/TLR4 (clone HTA125) purchased from Affymetrix.

Cytokine Quantification. Sandwich ELISAs were performed using capture and biotinylated antibodies for human resistin (Peprotech), IL-10 and TNFα (BD Biosciences) according to manufacturer's instructions. Detection was performed with streptavidin-peroxidase (Jackson ImmunoResearch) and TMB peroxidase substrate (BD Biosciences), followed by addition of 2N H2SO4. Optical density (OD) was captured at 450 nm. Samples were compared to serial-fold dilution of recombinant protein. For Luminex, inflammatory cytokine kits (Affymetrix) were run according to manufacturer's instructions and quantified on Luminex MagPix (Luminex Corp.). For cytokine bead array, cytokine kits from BD Bioscience were run according to manufacturer's instructions on BD LSRII and analyzed using FCAP Array Software.

LAL assay. Recombinant hResistin was tested for endotoxin contamination using the Pierce Limulus Amebocyte Lysate (LAL) assay (Thermo Scientific) according to manufacturer's instructions under sterile conditions.

Pull-down Assay. 1 μg/ml protein His-tagged MBP, human TLR4 and human CAP1 were added to nickel-nitrilotriacetic acid-agarose beads (Invitrogen) in binding buffer (50 mM NaH2PO4, 500 mM NaCl, 10 mM Imidazole, pH 8.0) and mixed for 1 hour at 4° C., followed by washing and 1 h incubation at 4° C. with hRetn (E. coli-derived, Peprotech or 293T cell-derived, LifeSpan Biosciences) in binding buffer. Beads were washed in wash buffer (50 mM NaH2PO4, 500 mM NaCl, 20 mM Imidazole, pH 8.0), and complex protein eluted with elution buffer (50 mM NaH2PO4, 500 mM NaCl, 250 mM Imidazole, pH 6.0). His-MBP, His-TLR4 and His-CAP1 were detected by anti-His antibody (Abcam), and hRetn was detected by anti-hRetn antibody (donated by Mitchell Lazar) by Western Blot. For the N-peptide binding experiment, 1 μg/ml His-tagged human TLR4 with 500 ng/ml N-peptide or 10% TFE control was added to the beads in binding buffer and mixed for 30 min at 4° C., followed by co-immunoprecipitation using 1 μg/ml Flag-hRetn (LifeSpan Biosciences), anti-His or anti-Flag (Sigma).

hRetn N-terminal Peptide Synthesis. hRetn N-terminal peptide (1-23a.a.) (SEQ ID NO: 2) were synthesized using a microwave assisted solid-phase synthesizer (Liberty Blue, CEM Corp.) and a double coupling protocol. The agent was subsequently purified by reverse phase HPLC and characterized by HRMS (high resolution mass spectrometry) and NMR (nuclear magnetic resonance). The soluble hRetn N-peptide helical content was determined by circular dichroism measurements in aqueous buffer containing 10% TFE (trifluoroethanol).

Human FcIg-resistin expression. A plasmid was designed for human FcIg-resistin fusion protein expression in Drosophila S2 cells (pMT/V5 His-TOPO, Invitrogen), see FIG. 7A. In brief, S2 cells were transfected with the plasmid and successful transfectants were selected by hygromycin resistance conferred by co-transfected hygromycin-resistant plasmid (pCoHygro selection vector). A stable transfection line was obtained after 2 week-1 month culture, and fusion protein expression was induced by CuSO₄ for one week. Purification of the fusion protein was performed by Protein A column (Invitrogen).

Signaling Western Blot. 1×10⁶ peritoneal cells from one mouse or 3×10⁶ cell pooled from 3 mice per group were lysed in RIPA buffer (150 mM Sodium chloride, 1% Triton X-100, 1% Sodium deoxycholate, 0.1% Sodium dodecyl sulfate, 50 mM Tris-HCl and 2 mM EDTA). Proteins were boiled in loading buffer (Bioland Scientific LLC), denatured then separated with SDS-PAGE gels, transferred to PVDF membranes (Millipore), and blocked with 5% BSA (Sigma) or 5% milk. Signaling proteins were detected with the following antibodies: anti-pSTAT3 (Tyr705, Abcam), anti-pTBK1 (Ser172, clone D52C2), anti-IκBα, anti-β-actin, then incubated with anti-rabbit or mouse HRP-conjugated IgG. All antibodies were purchased from Cell Signaling Technology. Proteins were detected with ECL (Pierce Chemical Co.) and exposed with X-ray film or ChemiDoc™ XRS+System (Bio-Rad). For quantification of protein levels, appropriate film exposures were scanned and the density of bands was determined with Image J and normalized with endogenous β-actin.

Structural Predictions/Calculations. The structural analysis of the TLR4-resistin interactions was performed with the structure of human TLR4-MD2-LPS complex (PDB code: 3FXI) and human resistin built by homology modeling. The sequence of human resistin was downloaded from the universal protein sequence (Uniprot, Entry no. Q9HD89). Then, this sequence was used in the SWISS-Model Server for homology modeling to find a structural template. This server found murine resistin (PDB code: 1RFX) as the template with maximum sequence identity of 57.61%. Murine resistin 3D structure was used to build the structural model for human resistin using the server. The model of the trimer of human resistin was used perform docking studies with the human TLR4 using the ClusPro web server to predict potential interactions of the resistin trimer to the TLR4 dimer based on Van der Waal's electrostatic interactions. As control, models of the complex between MD2 and TLR4 were similarly generated and compared with the experimental X-ray structure of the complex, revealing an excellent agreement. PDBeP ISA server was used to confirm the feasibility of all the models and detailed protein-protein interactions. Pymol software was used for all modeling manipulation.

Statistical Analysis. All statistics were generated on GraphPad Prism using where appropriate log rank test, student t-test, one-way ANOVA or two-way ANOVA. ns, not significant; *, p≤0.05; **, p≤0.01; ***, p≤0.001; ****, p≤0.0001.

Ethics Statement. All protocols for animal use and euthanasia were approved by the University of California, Riverside Institutional Animal Care and Use Committee (https://or.ucr.edu/ori/committees/iacuc.aspx; protocol A-20150028E) and were in accordance with the National Institutes of Health Guidelines. Animal studies are in accordance with the provisions established by the Animal Welfare Act and the Public Health Servies (PHS) Policy on the Humane Care and Use of Laboratory Animals. Human buffy coat (˜60 mL of concentrated leukocytes and erythrocytes) were collected from healthy donors with signed informed consent by Zen-bio, Inc. Isolation of PBMC and assays were performed at UCR with the approval of the University of California, Riverside Institutional Review Board (HS-14-155, Exempt 4 category).

While the present disclosure has been illustrated and described with reference to certain exemplary embodiments, those of ordinary skill in the art will understand that various modifications and changes may be made to the described embodiments without departing from the spirit and scope of the present disclosure, as defined in the following claims.

What is claimed is:

1. A method of treating a Toll-like receptor 4 (TLR4)-mediated infection or disease in a host cell or in a subject having the TLR4-mediated infection or disease, the method comprising:
administering to the host cell or the subject an effective amount of a composition comprising a full length resistin protein, a resistin protein fragment, a fusion protein comprising a resistin protein or a fragment thereof and an immunoglobulin G protein, a fusion protein of an N-terminal or C-terminal resistin protein fragment and an immunoglobulin G (IgG) protein, homologs thereof, a derivative thereof, or a salt thereof.

2. The method of claim 1, wherein the TLR4-mediated infection or disease is selected from the group consisting of sepsis, acute respiratory distress syndrome (ARDS), rheumatoid arthritis, influenza, and traumatic injury.

3. The method of claim 1, wherein the TLR4-mediated infection or disease is sepsis.

4. The method of claim 1, wherein the full-length resistin protein, the resistin protein fragment, the fusion protein comprising the resistin protein or the fragment thereof and the immunoglobulin G protein, the fusion protein of an N-terminal or C-terminal resistin protein fragment or the IgG protein has a human or mouse amino acid sequence.

5. The method of claim 1, wherein the resistin protein fragment comprises at least one of Chain M, Chain H, or Chain C of human resistin protein (SEQ ID NO: 1).

6. The method of claim 5, wherein the resistin protein fragment is a monomer or trimer.

7. The method of claim 1, wherein the N-terminal resistin protein fragment comprises any consecutive 84 amino acids of human resistin protein (hRetn) (SEQ ID NO: 1) or homologs thereof.

8. The method of claim 7, wherein the consecutive 84 amino acids comprise at least residues 24 to 46 (SEQ ID NO: 2) or homologs thereof.

9. The method of claim 1, wherein the C-terminal resistin protein fragment comprises residues 51 to 108 (SEQ ID NO: 4) or homologs thereof.

10. The method of claim 1, wherein the IgG protein is a fragment crystallizable (Fc) region of the IgG protein.

11. The method of claim 10, wherein the Fc region has an amino acid sequence of SEQ ID NO: 5 or homologs thereof.

12. The method of claim 1, wherein the resistin protein of the fusion protein comprising the resistin protein or the fragment thereof and the IgG protein, comprises a human resistin protein (hRetn) (SEQ ID NO: 1), any consecutive 84 amino acids of hRetn (SEQ ID NO: 1), or homologs thereof.

13. The method of claim 1, wherein the composition further comprises a pharmaceutical carrier.

14. A composition comprising a fragment of the full-length resistin protein, the fragment comprising any consecutive 84 amino acids of the human resistin protein hRetn (SEQ ID NO: 1) or homologs thereof.

15. The composition of claim 14, wherein the consecutive 80 amino acids comprise at least residues 24 to 46 (SEQ ID NO: 2) or homologs thereof.

16. The composition of claim 14, wherein the fragment of the full-length resistin protein forms a fusion protein with an immunoglobulin G (IgG).

17. The composition of claim 16, wherein the fragment comprises at least residues 24 to 46 (SEQ ID NO: 2), residues 24 to 50 (SEQ ID NO: 3), or homologs thereof, and the IgG protein is a fragment crystallizable (Fc) region of the IgG (Fc IgG) protein having an amino acid sequence of SEQ ID NO: 5 or homologs thereof.

18. The composition of claim 17, wherein the fragment comprises residues 51 to 108 of the human resistin protein (SEQ ID NO: 1) or homologs thereof.

19. A composition comprising a fusion protein of human resistin protein SEQ ID NO: 1 or homologs thereof and a fragment crystallizable (Fc) region of the IgG protein (Fc IgG) having an amino acid sequence of SEQ ID NO: 5.
